(19) United States
(12) Patent Application Publication
(10) Pub. No.: US 2007/0191599 A1
(43) Pub. Date: Aug. 16, 2007

(54) METHODS OF MANUFACTURING CORTISCOSTEROID SOLUTIONS

(75) Inventors: Malcolm Hill, Solana Beach, CA (US); Cynthia LiCalsi, San Diego, CA (US); Troy Christensen, Mason, OH (US)

Correspondence Address: WILSON SONSINI GOODRICH & ROSATI, 650 PAGE MILL ROAD, PALO ALTO, CA, 94304-1050, US

(73) Assignee: VERUS PHARMACEUTICALS, INC., San Diego, CA (US)

(21) Appl. No.: 11/675,569

(22) Filed: Feb. 15, 2007

Related U.S. Application Data
(60) Provisional application No. 60/774,151, filed on Feb. 15, 2006.
(60) Provisional application No. 60/774,073, filed on Feb. 15, 2006.
(60) Provisional application No. 60/774,152, filed on Feb. 15, 2006.

(51) Int. Cl. A61K 31/58; A61K 31/573

(52) U.S. Cl. 540/63; 552/503; 514/179; 514/171

(57) ABSTRACT

The present invention relates to methods of manufacturing compositions comprising a corticosteriod and at least one solubility enhancer, as well as compositions made by these methods.

### PRIORITY CLAIM AND CROSS-REFERENCE TO RELATED APPLICATIONS

This application claims the benefit of and priority under 35 U.S.C. § 119(e) from U.S. Provisional Patent Application No. 60/774,151, which was filed on Feb. 15, 2006, and which is incorporated herein by reference in its entirety. This application further claims the benefit of and priority under 35 U.S.C. 119(e) to U.S. provisional patent application 60/774,073, filed on Feb. 15, 2006, which is incorporated herein by reference in its entirety. This application further claims the benefit of and priority under 35 U.S.C. § 119(e) from U.S. Provisional Patent Application No. 60/774,152, filed on Feb. 15, 2006, which is incorporated herein by reference in its entirety.

This application is related to copending application Ser. No. 11/675,563, filed Feb. 15, 2007, entitled “Sterilization of Corticosteroids With Reduced Mass Loss,” Attorney Docket Number 31622-717/201, which is incorporated herein by reference in its entirety. This application is also related to copending application Ser. No. 11/675,575, filed Feb. 15, 2007, entitled “Stable Corticosteroid Mixtures,” Attorney Docket Number 31622-719/201, which is incorporated herein by reference in its entirety.

### FIELD OF THE INVENTION

The present invention relates to methods of manufacturing compositions comprising a corticosteroid and at least one solubility enhancer, as well as compositions made by these methods.

### BACKGROUND OF THE INVENTION

Inhaled corticosteroids are fundamental to the long-term management of respiratory diseases such as CPOD and persistent asthma and are recommended by national guidelines for therapy of young children diagnosed with asthma. Numerous clinical trials support their efficacy and relative safety for children. In addition, it is believed that early corticosteroid intervention can play a critical role in the reduction of permanent lung damage and alter the chronic, progressive nature of the disease.

The use of inhaled corticosteroids in the treatment of asthma provides significant benefit due to the direct delivery to the site of action, the lung (as used herein, “lung” refers to either or both the right and left lung organs). The goal of inhaled corticosteroid therapy is to provide localized delivery of the corticosteroid with immediate drug activity at the site of action. It is known that inhaled corticosteroids are well absorbed from the lungs. In fact, it can be assumed that substantially all of the drug available at the receptor site in the lungs will be absorbed. However, it is also known that current methods and formulations result in a greater part of an inhaled corticosteroid dose being swallowed and becoming available for oral adsorption. Thus, due to the particular method or system employed, some corticosteroids are more likely to be deposited in the mouth and throat than the lungs, and may cause adverse effects. For the portion of the inhaled corticosteroid dose delivered orally, bioavailability depends upon absorption from the GI tract and the extent of first pass metabolism in the liver. Since this oral component of corticosteroid drug delivery does not provide any beneficial therapeutic effect and increases the risk of systemic side effects, it is desirable for the oral bioavailability of inhaled corticosteroid to be relatively low. Thus, for inhaled corticosteroids, high pulmonary availability is more important than high oral bioavailability because the lung is the target organ.

Budesonide (R,S)-11β,16α,17,21-tetrahydroxypregna-1,4-diene-3,20-dione cyclic 16,17-acetal with butyraldehyde, (C₂H₃₄O₆; MW: 430.5) is employed in particular for the treatment of bronchial disorders. Budesonide is a racemate consisting of a mixture of the two diastereomers 22R and 22S and is provided commercially as a mixture of the two isomers (22R and 22S). It acts as an anti-inflammatory corticosteroid that exhibits potent glucocorticoid activity. Administration of budesonide is indicated for maintenance treatment of asthma and as prophylactic therapy in children.

The manufacture of corticosteroid (e.g. budesonide) solutions is hampered at least in part by the poor wetability, low solubility and slow dissolution of corticosteroid particles. One result of the poor wetability is that corticosteroid tends to clump when added to a dissolution container. Although improvements in the equilibrium solubility of corticosteroids such as budesonide can be achieved using cyclodextrins as solubility enhancers, it has remained difficult to achieve timely wetting and dissolution of corticosteroid, due to the poor wetability, and concomitant clumping, of corticosteroid. There is thus a need for a process that avoids this difficulty caused by the poor wetability of corticosteroids low solubility and slow dissolution, such as budesonide.

### SUMMARY OF THE INVENTION

Provided herein are methods of making a corticosteroid solution comprising the steps of: (a) combining ingredients of the corticosteroid solution comprising as starting materials a corticosteroid, at least one solubility enhancer and water in a high sheer mixer; and (b) homogenizing the ingredients for a homogenizing period; whereby at least about 95% of the corticosteroid starting material is dissolved within the homogenizing period.

Also provided herein are methods of making a corticosteroid solution comprising the steps of: (a) combining ingredients of the corticosteroid solution comprising as starting materials a corticosteroid, at least one solubility enhancer and water in a high sheer mixer having a capacity greater than about 5 L; and (b) homogenizing the ingredients for a homogenizing period of about 2 hours or less; whereby at least about 98% of the corticosteroid starting material is dissolved within the homogenizing period.

Provided herein are also methods of making a corticosteroid solution comprising the steps of: (a) combining ingredients of the corticosteroid solution comprising as starting materials a corticosteroid, at least one solubility enhancer and water in a high sheer mixer having a capacity greater than or equal to about 50 L; and (b) homogenizing the ingredients for a homogenizing period of about 5 hours or less; whereby at least about 98% of the corticosteroid starting material is dissolved within the homogenizing period.

Provided herein are also methods of making a budesonide solution comprising the steps of: (a) combining ingredients of the budesonide solution comprising as starting materials budesonide, a cyclodextrin solubility enhancer and water in a high sheer mixer having a capacity greater than or equal to about 50 L; and (b) homogenizing the ingredients for a homogenizing period of about 5 hours or less; whereby at least about 98% of the budesonide is dissolved within the homogenizing period. In some preferred embodiments of the invention, the high sheer mixer has a capacity of 100 L or greater. In some preferred embodiments of the invention, the high sheer mixer has a capacity of 200 L or greater. In some preferred embodiments of the invention, the high sheer mixer has a capacity of 300 L or greater. In some preferred embodiments of the invention, the high sheer mixer has a capacity of 400 L or greater. In some preferred embodiments of the invention, the high sheer mixer has a capacity of 500 L or greater. In some preferred embodiments of the invention, the high sheer mixer has a capacity of 1000 L, 4000 L, 10,000 L or greater.

Provided herein are also methods of making a budesonide solution comprising the steps of: (a) combining ingredients of the budesonide solution comprising as starting materials budesonide, a cyclodextrin solubility enhancer and water in a high sheer mixer having a capacity of between about 50 L and about 10,000 L or more; and (b) homogenizing the ingredients for a homogenizing period of about 5 hours or less; whereby at least about 98% of the budesonide is dissolved within the homogenizing period. In some embodiments, the high sheer mixer has a capacity of between about 50 L and 10,000 L, especially between about 100 L and 10,000 L, particularly between about 200 L and 1000 L, between about 300 L and 1000 L and from about 500 L to about 1000 L.

In certain embodiments of the present invention, the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE-γ-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In other embodiments, the corticosteroid is budesonide.

In some embodiments, the solubility enhancer is a sulfoalkyl ether cyclodextrin (SAE-CD). In preferred embodiments, the solubility enhancer is SBE7-β-CD (e.g. Captisol®, CyDex).

In some embodiments, the corticosteroid solution or budesonide solution further comprises albuterol.

In various embodiments, at least about 95%, at least about 97%, at least about 98%, or at least about 99% of the corticosteroid is dissolved within the homogenizing period.

In some embodiments, the homogenizing period is about 3 days, about 2 days, about 1 day, about 18 hours, about 12 hours, about 6 hours, about 5 hours, about 4 hours, about 3 hours, about 2 hours, about 1 hour, about 45 minutes, about 30 minutes, or about 15 minutes.

In various embodiments, at least about 95%, at least about 97%, or at least about 99% of the corticosteroid is dissolved within the first hour of the homogenizing period.

In some embodiments, the high sheer mixer has a capacity of about 5 L to about 2000 L, about 250 L to about 750 L, about 100 L to about 1000 L, or about 50 L to 500 L.

In other embodiments, the high sheer mixer has a capacity of about 5 L, about 10 L, about 20 L, about 30 L, about 40 L, about 50 L, about 75 L, about 100 L, about 125 L, about 150 L, about 175 L, about 200 L, about 250 L, about 300 L, about 350 L, about 400 L, about 450 L, about 500 L, about 750 L, about 1000 L, about 1500 L, or about 2000 L.

In various embodiments, the volume of the corticosteroid solution is about 5 L, about 10 L, about 20 L, about 30 L, about 40 L, about 50 L, about 75 L, about 100 L, about 125 L, about 150 L, about 175 L, about 200 L, about 250 L, about 300 L, about 350 L, about 400 L, about 450 L, about 500 L, about 750 L, about 1000 L, about 1500 L, or about 2000 L.

In some embodiments, the corticosteroid solution comprises a combination of two or more solubility enhancers. In some embodiments the solubility enhancer is a combination of a cyclodextrin and a polyoxyethylene sorbitan monooleate such as polysorbate 80 (PS 80). In various embodiments, the polysorbate is present in an amount of between about 0.005 wt-% to about 0.1 wt-%. In other embodiments, the corticosteroid solution substantially excludes polysorbate. In yet other embodiments, the corticosteroid solution contains less than about 0.01 wt-% polysorbate or less than about 0.005 wt-% polysorbate.

In some embodiments, the high sheer mixer is a FrymaKoruma Dinex model 700, 1300, 2400, 3500, 4200 or 5200 (FrymaKoruma GmbH, Neuenburg, Germany).

In other embodiments, the homogenization speed is between about 500 to about 5000 rpm, about 1000 to about 3000 rpm, or about 1500 to about 2000 rpm.

In some embodiments, the invention provides a process of making a corticosteroid solution, comprising the steps of: (a) combining ingredients of the corticosteroid solution comprising as starting materials a corticosteroid, at least one solubility enhancer and water in a high sheer mixer having a capacity greater than or equal to about 50 L; and (b) homogenizing the ingredients for a homogenizing period of about 5 hours or less; whereby at least about 98% of the corticosteroid starting material is dissolved within the homogenizing period. In some embodiments, the corticosteroid is budesonide. In some embodiments, the solubility enhancer comprises a sulfoalkyl ether cyclodextrin (SAE-CD), such as SAE-CD is SBE7-β-CD. In some embodiments, the corticosteroid solution further comprises albuterol. In some embodiments, at least about 98.5% of the corticosteroid is dissolved within the homogenizing period. In some embodiments, the homogenizing period is about 2 hours or less. In some embodiments, at least about 99% of the corticosteroid is dissolved within the homogenizing period. In some embodiments, the homogenizing period is about 2 hours or less. In some embodiments, at least about 99.5% of the corticosteroid is dissolved within the homogenizing period. In some embodiments, the homogenizing period is about 2 hours or less. In some embodiments, at least about 95% of the corticosteroid is dissolved within the first hour of the homogenizing period. In some embodiments, at least about 97% of the corticosteroid is dissolved within the first hour of the homogenizing period. In some embodiments, the high sheer mixer has a capacity of about 100 L to about 1000 L. In some embodiments, the high sheer mixer has a capacity of about 250 L to about 750 L. In some embodiments, the high sheer mixer has a capacity of about 500 L. In some embodiments, the budesonide solution substantially excludes polysorbate 80. In some embodiments, the budesonide solution contains less than about 0.01 wt-% polysorbate 80. In some embodiments, the budesonide solution contains less than about 0.005 wt-% polysorbate 80. In some embodiments, the budesonide solution comprises two or more solubility enhancers. In some embodiments, the solubility enhancer is a combination of polyoxyethylene sorbitan monooleate and a cyclodextrin. In some embodiments, the polyoxyethylene sorbitan monooleate is polysorbate 80. In some embodiments, the polysorbate is present in an amount of between about 0.005 wt-% to about 0.1 wt-%. In some embodiments, the high sheer mixer is a FrymaKoruma Dinex model 700, 1300, 2400, 3500, 4200 or 5200. In some embodiments, the high sheer mixer is a FrymaKoruma Dinex model 700. In some embodiments, the homogenization speed is between about 1000 to about 3000 rpm. In some embodiments, the process comprises homogenizing the mixture at a homogenization speed of about 1500 to about 3000 rpm. In some embodiments, the homogenization speed is about 1700 rpm to about 2500 rpm.

In some embodiments, the invention provides a process of making a corticosteroid solution, comprising the steps of: (a) combining ingredients of the corticosteroid solution comprising as starting materials a corticosteroid, at least one solubility enhancer and water in a high sheer mixer having a capacity greater than or equal to about 50 L; and (b) homogenizing the ingredients for a homogenizing period of about 2 hours or less; whereby at least about 98% of the corticosteroid starting material is dissolved within the homogenizing period. In some embodiments, the corticosteroid is budesonide. In some embodiments, the solubility enhancer comprises a sulfoalkyl ether cyclodextrin (SAE-CD). In some embodiments, the SAE-CD is SBE7-β-CD. In some embodiments, the corticosteroid solution further comprises albuterol. In some embodiments, at least about 98.5% of the corticosteroid is dissolved within the homogenizing period. In some embodiments, the homogenizing period is about 2 hours or less. In some embodiments, at least about 99% of the corticosteroid is dissolved within the homogenizing period. In some embodiments, the homogenizing period is about 2 hours or less. In some embodiments, at least about 99.5% of the corticosteroid is dissolved within the homogenizing period. In some embodiments, the homogenizing period is about 2 hours or less. In some embodiments, at least about 95% of the corticosteroid is dissolved within the first hour of the homogenizing period. In some embodiments, at least about 97% of the corticosteroid is dissolved within the first hour of the homogenizing period. In some embodiments, the high sheer mixer has a capacity of about 100 L to about 1000 L. In some embodiments, the high sheer mixer has a capacity of about 250 L to about 750 L. In some embodiments, the high sheer mixer has a capacity of about 500 L. In some embodiments, the budesonide solution substantially excludes polysorbate 80. In some embodiments, the budesonide solution contains less than about 0.01 wt-% polysorbate 80. In some embodiments, the budesonide solution contains less than about 0.005 wt-% polysorbate 80. In some embodiments, the budesonide solution comprises two or more solubility enhancers. In some embodiments, the solubility enhancer is a combination of polyoxyethylene sorbitan monooleate and a cyclodextrin. In some embodiments, the polyoxyethylene sorbitan monooleate is polysorbate 80. In some embodiments, the polysorbate is present in an amount of between about 0.005 wt-% to about 0.1 wt-%. In some embodiments, the high sheer mixer is a FrymaKoruma Dinex model 700, 1300, 2400, 3500, 4200 or 5200. In some embodiments, the high sheer mixer is a FrymaKoruma Dinex model 700. In some embodiments, the homogenization speed is between about 1000 to about 3000 rpm. In some embodiments, homogenizing the mixture at a homogenization speed of about 1500 to about 3000 rpm. In some embodiments, the homogenization speed is about 1700 rpm to about 2500 rpm.

In some embodiments, the invention provides a process of making a budesonide solution, comprising the steps of: (a) combining ingredients of the budesonide solution comprising as starting materials budesonide, a cyclodextrin solubility enhancer and water in a high sheer mixer having a capacity greater than or equal to about 100 L; and (b) homogenizing the ingredients for a homogenizing period of about 2 hours or less; whereby at least about 98% of the corticosteroid starting material is dissolved within the homogenizing period. In some embodiments, the cyclodextrin solubility enhancer is a sulfoalkyl ether cyclodextrin (SAE-CD). In some embodiments, the SAE-CD is SBE7-β-CD. In some embodiments, the corticosteroid solution further comprises albuterol. In some embodiments, at least about 98.5% of the corticosteroid is dissolved within the homogenizing period. In some embodiments, the homogenizing period is about 2 hours or less. In some embodiments, at least about 99% of the corticosteroid is dissolved within the homogenizing period. In some embodiments, the homogenizing period is about 2 hours or less. In some embodiments, at least about 99.5% of the corticosteroid is dissolved within the homogenizing period. In some embodiments, the homogenizing period is about 2 hours or less. In some embodiments, at least about 95% of the corticosteroid is dissolved within the first hour of the homogenizing period. In some embodiments, at least about 97% of the corticosteroid is dissolved within the first hour of the homogenizing period. In some embodiments, the high sheer mixer has a capacity of about 100 L to about 1000 L. In some embodiments, the high sheer mixer has a capacity of about 250 L to about 750 L. In some embodiments, the high sheer mixer has a capacity of about 500 L. In some embodiments, the budesonide solution substantially excludes polysorbate 80. In some embodiments, the budesonide solution contains less than about 0.01 wt-% polysorbate 80. In some embodiments, the budesonide solution contains less than about 0.005 wt-% polysorbate 80. In some embodiments, the budesonide solution comprises two or more solubility enhancers. In some embodiments, the solubility enhancer is a combination of polyoxyethylene sorbitan monooleate and a cyclodextrin. In some embodiments, the polyoxyethylene sorbitan monooleate is polysorbate 80. In some embodiments, the polysorbate is present in an amount of between about 0.005 wt-% to about 0.1 wt-%. In some embodiments, the high sheer mixer is a FrymaKoruma Dinex model 700, 1300, 2400, 3500, 4200 or 5200. In some embodiments, the high sheer mixer is a FrymaKoruma Dinex model 700. In some embodiments, the homogenization speed is between about 1000 to about 3000 rpm. In some embodiments, the mixture at a homogenization speed of about 1500 to about 2000 rpm. In some embodiments, the homogenization speed is about 1700 rpm to about 2500 rpm.

In some embodiments, the invention provides a process of making a corticosteroid solution, comprising the steps of: (a) combining ingredients of the corticosteroid solution comprising as starting materials a corticosteroid, at least one solubility enhancer and water in a high sheer mixer; and (b) homogenizing the ingredients for a homogenizing period; whereby at least about 95% of the corticosteroid starting material is dissolved within the homogenizing period. In some embodiments, the corticosteroid is budesonide. In some embodiments, the solubility enhancer is a sulfoalkyl ether cyclodextrin (SAE-CD). In some embodiments, the SAE-CD is SBE7-β-CD. In some embodiments, the corticosteroid solution further comprises albuterol. In some embodiments, the homogenizing period is about 3 days, about 2 days, about 1 day, about 18 hours, about 12 hours, or about 6 hours. In some embodiments, the homogenizing period is about 5 hours, about 4 hours, about 3 hours, about 2 hours, about 1 hour, about 45 minutes, about 30 minutes, or about 15 minutes. In some embodiments, at least about 95% of the corticosteroid is dissolved within the homogenizing period. In some embodiments, at least about 97% of the corticosteroid is dissolved within the homogenizing period. In some embodiments, at least about 99% of the corticosteroid is dissolved within the homogenizing period. In some embodiments, at least about 95%, or about 97%, or about 99% of the corticosteroid is dissolved within the first hour of the homogenizing period. In some embodiments, the high sheer mixer has a capacity of about 5 L to about 2000 L, about 250 L to about 750 L, about 100 L to about 1000 L, or about 50 L to 500 L. In some embodiments, the high sheer mixer has a capacity of about 5 L, about 10 L, about 20 L, about 30 L, about 40 L, about 50 L, about 75 L, about 100 L, about 125 L, about 150 L, about 175 L, about 200 L, about 250 L, about 300 L, about 350 L, about 400 L, about 450 L, about 500 L, about 750 L, about 1000 L, about 1500 L, or about 2000 L. In some embodiments, the budesonide solution substantially excludes polysorbate 80. In some embodiments, the budesonide solution contains less than about 0.01 wt-% polysorbate 80. In some embodiments, the budesonide solution contains less than about 0.005 wt-% polysorbate 80. In some embodiments, the budesonide solution comprises two or more solubility enhancers. In some embodiments, the solubility enhancer is a combination of polyoxyethylene sorbitan monooleate and a cyclodextrin. In some embodiments, the polyoxyethylene sorbitan monooleate is polysorbate 80. In some embodiments, the polysorbate is present in an amount of between about 0.005 wt-% to about 0.1 wt-%. In some embodiments, the high sheer mixer is a FrymaKoruma Dinex model 700, 1300, 2400, 3500, 4200 or 5200. In some embodiments, the homogenization speed is between about 500 to about 5000 rpm, about 1000 to about 3000 rpm, or about 1500 to about 2000 rpm.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### INCORPORATION BY REFERENCE

All publications and patent applications mentioned in this specification are herein incorporated by reference to the same extent as if each individual publication or patent application was specifically and individually indicated to be incorporated by reference.

### DESCRIPTION OF THE FIGURES

FIG. 1 shows the dissolution rate of the corticosteroid, budesonide, with varying amounts of Captisol® (SBE7-β-CD) with and without PS80. The procedures for the studies are described in Examples 1A-1D.

FIG. 2 shows the dissolution rate of the corticosteroid, budesonide, with varying amounts of Captisol® (SBE7-β-CD). The procedures for the studies are described in Examples 1A-1C.

FIG. 3 shows the dissolution rate of the corticosteroid, budesonide, with varying amounts of Captisol® (SBE7-β-CD) with and without PS80. The procedures for the studies are described in Examples 1A-1D.

FIG. 4 is a process flow diagram including process steps according to the present invention.

FIG. 5 is a process flow diagram depicting an alternative embodiment of the dissolution process according to the present invention.

FIG. 6 is a graph demonstrating the effect of temperature on the dissolution profiles of two concentrations of budesonide solution.

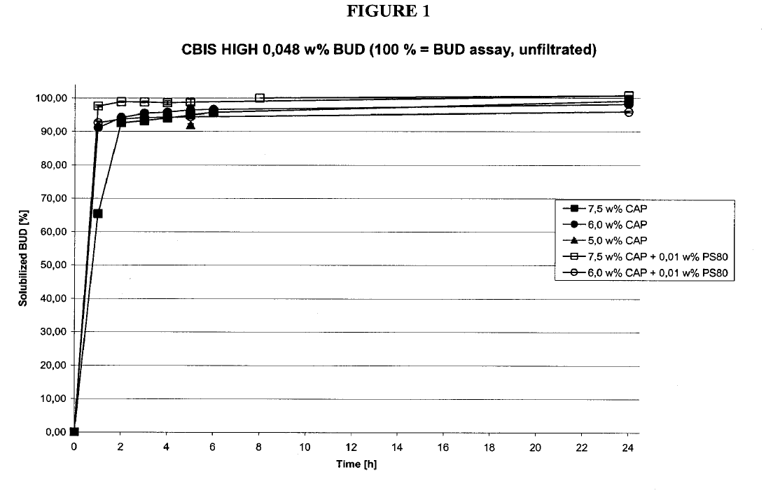

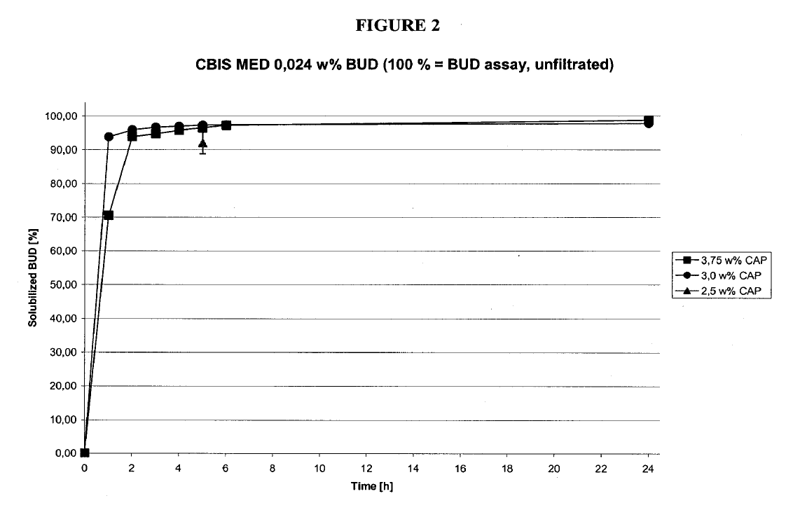

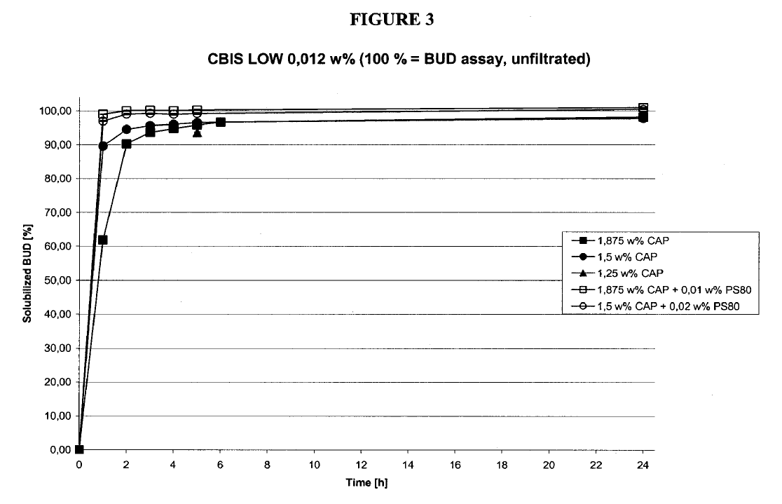

### DETAILED DESCRIPTION OF THE INVENTION

As mentioned above, the poor wetability of corticosteroids such as budesonide has made it difficult to prepare corticosteroid solutions, e.g. due to the tendency of the corticosteroid starting materials to clump when combined with water. While the overall solubility of corticosteroids such as budesonide have been improved with the use of cyclodextrins as solubility enhancers, dissolution of corticosteroids such as budesonide has been slow. Micronized corticosteroids, such as micronized budesonide, provide an improvement in dynamic dissolution profile. The present invention provides a solution to the problem of poor corticosteroid wetability by providing a process in which corticosteroid such as budesonide is introduced into a high speed mixer under high sheer conditions. The high sheer conditions of the mixer quickly wet the corticosteroid particles (e.g. budesonide microparticles), causing them to be suspended in the aqueous solvent before they have a chance to agglomerate (clump). Surprisingly, it has also been found that using at batch sizes of about 50 L and larger according to the present invention, the dissolution profile of a corticosteroid such as budesonide is greatly improved over the dissolution profiles of smaller batch sizes—e.g. on the order of about 10 L or less.

Reference will now be made in detail to certain illustrative and non-limiting embodiments of the compositions and methods disclosed herein. Examples of the embodiments are illustrated in the following Examples section.

Unless defined otherwise, all technical and scientific terms used herein have the same meaning as is commonly understood by one of skill in the art to which the inventions described herein belong. All patents and publications referred to herein are incorporated by reference.

### Certain Definitions

As used herein, the terms “comprising,” “including,” “such as,” and “for example” are used in their open, non-limiting sense.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe.

A “therapeutically effective amount” or “effective amount” is that amount of a pharmaceutical agent to achieve a pharmacological effect. The term “therapeutically effective amount” includes, for example, a prophylactically effective amount. An “effective amount” of a corticosteroid, such as budesonide, is an amount effective to achieve a desired pharmacologic effect or therapeutic improvement without undue adverse side effects. The effective amount of a corticosteroid, such as budesonide, will be selected by those skilled in the art depending on the particular patient and the disease level. It is understood that “an effective amount” or “a therapeutically effective amount” can vary from subject to subject, due to variation in metabolism of a corticosteroid, such as budesonide, age, weight, general condition of the subject, the condition being treated, the severity of the condition being treated, and the judgment of the prescribing physician.

“Treat” or “treatment” as used in the context of a bronchoconstrictive disorder refers to any treatment of a disorder or disease related to the contraction of the bronchi, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

I. Corticosteroids

The term “corticosteroid” is intended to have the full breadth understood by those of skill in the art. Particular corticosteroids contemplated within the scope of the invention are those that are not generally soluble in water to a degree suitable for pharmaceutical administration, and thus require the presence of at least one solubility enhancer to dissolve them in aqueous solution. Particular corticosteroids that may be mentioned in this regard include those set forth in WO 2005/065649, WO 2005/065435 and WO 2005/065651. See in particular page 46 of WO 2005/065651, which is incorporated herein by reference.

The corticosteroids that may be substituted for budesonide include aldosterone, beclomethasone, betamethasone, ciclesonide, cloprednol, cortisone, cortivazol, deoxycortone, desonide, desoximetasone, dexamethasone, difluorocortolone, fluclorolone, flumethasone, flunisolide, flucinolone, fluocinonide, fluocortin butyl, fluocortisone, flurocortolone, fluorometholone, flurandrenolone, fluticasone, halcinonide, hydrocortisone, icomethasone, meprednisone, methylpredinsolone, mometasone, paramethasone, prednisolone, prednisone, rofleponide, RPR 106541, tixocortol, triamcinolone and their pharmaceutically active derivatives, including prodrugs and pharmaceutically acceptable salts.

In some embodiments, the corticosteroid is budesonide. In other embodiments, the corticosteroid is budesonide wherein the budesonide is either an individual diastereomer or a mixture of the two diastereomers administered individually or together for a therapeutic effect. In preferred embodiments, the budesonide is micronized budesonide.

In some embodiments, the corticosteroid is micronized (e.g. micronized budesonide).

The weight % of corticosteroid in the corticosteroid solutions of the present invention may vary, including from about 0.001 to about 1. In some embodiments, the wt-% of corticosteroid in the corticosteroid solution is between about 0.001 to about 0.1, or between about 0.005 to about 0.1, or between about 0.005 to about 0.05 wt-%.

The concentration of corticosteroid in the corticosteroid solutions of the present invention may vary, including from about 1 μg/ml to about 2000 μg/ml. Particular values that may be mentioned are about 1, about 5, about 10, about 20, about 50, about 100, about 200, about 300, about 400, about 500, about 600, about 700, about 800, about 900, about 1000, about 1500, and about 2000 μg/ml. In some embodiments, the corticosteroid in the corticosteroid solution of the present invention is between about 50 to about 1000 μg/ml, or between about 100 to about 800 μg/ml, or between about 200 to about 600 μg/ml. In some embodiments, concentrations of 80 μg/mL, 120 μg/mL, 240 μg/mL and 480 μg/mL of budesonide are preferred.

II. Solubility Enhancers

The term “solubility enhancer” is intended to have the full breadth understood by those of skill in the art.

Solubility enhancers are known in the art and are described in, e.g., U.S. Pat. Nos. 5,134,127, 5,145,684, 5,376,645, 6,241,969 and U.S. Pub. Appl. Nos. 2005/0244339 and 2005/0008707, each of which is specifically incorporated by reference herein. In addition, examples of suitable solubility enhancers are described below.

Solubility enhancers suitable for use in the present invention include, but are not limited to, propylene glycol, non-ionic surfactants, phospholipids, cyclodextrins and derivatives thereof, and surface modifiers and/or stabilizers.

Examples of non-ionic surfactants which appear to have a particularly good physiological compatibility for use in the present invention are tyloxapol, polysorbates including, but not limited to, polyoxyethylene (20) sorbitan monolaurate, polyoxyethylene (20) sorbitan monopalmitate, polyoxyethylene (20) sorbitan monostearate (available under the tradename Tweens 20-40-60, etc.), Polysorbate 80, Polyethylene glycol 400; sodium lauryl sulfate; sorbitan laurate, sorbitan palmitate, sorbitan stearate (available under the tradename Span 20-40-60 etc.), benzalkonium chloride, PPO-PEO block copolymers (Pluronics), Cremophor-EL, vitamin E-TPGS (e.g., d-alpha-tocopheryl-polyethyleneglycol-1000-succinate), Solutol-HS-15, oleic acid PEO esters, stearic acid PEO esters, Triton-X100, Nonidet P-40, and macrogol hydroxystearates such as macrogol-15-hydroxystearate.

In some embodiments, the non-ionic surfactants suitable for use in the present invention are formulated with the corticosteroid to form liposome preparations, micelles or mixed micelles. Methods for the preparations and characterization of liposomes and liposome preparations are known in the art. Often, multi-lamellar vesicles will form spontaneously when amphiphilic lipids are hydrated, whereas the formation of small uni-lamellar vesicles usually requires a process involving substantial energy input, such as ultrasonication or high pressure homogenization. Further methods for preparing and characterizing liposomes have been described, for example, by S. Vemuri et al. (Preparation and characterization of liposomes as therapeutic delivery systems: a review. Pharm Acta Helv. 1995, 70(2):95-111) and U.S. Pat. Nos. 5,019,394, 5,192,228, 5,882,679, 6,656,497 each of which is specifically incorporated by reference herein.

In some cases, for example, micelles or mixed micelles may be formed by the surfactants, in which poorly soluble active agents can be solubilized. In general, micelles are understood as substantially spherical structures formed by the spontaneous and dynamic association of amphiphilic molecules, such as surfactants. Mixed micelles are micelles composed of different types of amphiphilic molecules. Both micelles and mixed micelles should not be understood as solid particles, as their structure, properties and behavior are much different from solids. The amphiphilic molecules which form the micelles usually associate temporarily. In a micellar solution, there is a dynamic exchange of molecules between the micelle-forming amphiphile and monomolecularly dispersed amphiphiles which are also present in the solution. The position of the drug molecules which are solublized in such micelles or mixed micelles depends on the structure of these molecules as well as the surfactants used. For example, it is to be assumed that particularly non-polar molecules are localized mainly inside the colloidal structures, whereas polar substances are more likely to be found on the surface. In one embodiment of a micellar or mixed micellar solution, the average size of the micelles may be less than about 200 nm (as measured by photon correlation spectroscopy), such as from about 10 nm to about 100 nm. Particularly preferred are micelles with average diameters of about 10 to about 50 nm. Methods of producing micelles and mixed micelles are known in the art and described in, for example, U.S. Pat. Nos. 5,747,066 and 6,906,042, each of which is specifically incorporated by reference herein.

Phospholipids are defined as amphiphile lipids which contain phosphorus. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols, etc. Lecithins are natural mixtures of various phospholipids which usually have a high proportion of phosphatidyl cholines. Depending on the source of a particular lecithin and its method of extraction and/or enrichment, these mixtures may also comprise significant amounts of sterols, fatty acids, tryglycerides and other substances.

Additional phospholipids which are suitable for use in the present invention on account of their physiological properties comprise, in particular, phospholipid mixtures which are extracted in the form of lecithin from natural sources such as soja beans (soy beans) or chickens egg yolk, preferably in hydrogenated form and/or freed from lysolecithins, as well as purified, enriched or partially synthetically prepared phospholipids, preferably with saturated fatty acid esters. Of the phospholipid mixtures, lecithin is particularly preferred. The enriched or partially synthetically prepared medium- to long-chain zwitterionic phospholipids are mainly free of unsaturations in the acyl chains and free of lysolecithins and peroxides. Examples for enriched or pure compounds are dimyristoyl phosphatidyl choline (DMPC), distearoyl phosphatidyl choline (DSPC) and dipalmitoyl phosphatidyl choline (DPPC). Of these, DMPC is currently more preferred. Alternatively, phospholipids with oleyl residues and phosphatidyl glycerol without choline residue are suitable for some embodiments and applications of the invention.

In some embodiments, the non-ionic surfactants and phospholipids suitable for use in the present invention are formulated with the corticosteroid to form colloidal structures. Colloidal solutions are defined as mono-phasic systems wherein the colloidal material dispersed within the colloidal solution does not have the measurable physical properties usually associated with a solid material. Methods of producing colloidal dispersions are known in the art, for example as described in U.S. Pat. No. 6,653,319, which is specifically incorporated by reference herein.

Suitable cyclodextrins and derivatives for use in the present invention are described in the art, for example, Challa et al., AAPS PharmSciTech 6(2): E329-E357 (2005), U.S. Pat. Nos. 5,134,127, 5,376,645, 5,874,418, each of which is specifically incorporated by reference herein. In some embodiments, suitable cyclodextrins or cyclodextrin derivatives for use in the present invention include, but are not limited to, α-cyclodextrins, β-cyclodextrins, γ-cyclodextrins, SAE-CD derivatives (e.g., SBE-α-CD, SBE-β-CD (Captisol®), and SBE-γ-CD) (CyDex, Inc. Lenexa, Kans.), hydroxyethyl, hydroxypropyl (including 2- and 3-hydroxypropyl) and dihydroxypropyl ethers, their corresponding mixed ethers and further mixed ethers with methyl or ethyl groups, such as methylhydroxyethyl, ethyl-hydroxyethyl and ethyl-hydroxypropyl ethers of α-, β- and γ-cyclodextrin; and the maltosyl, glucosyl and maltotriosyl derivatives of α-, β- and γ-cyclodextrin, which may contain one or more sugar residues, e.g. glucosyl or diglucosyl, maltosyl or dimaltosyl, as well as various mixtures thereof, e.g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Procedures for preparing such cyclodextrin derivatives are well-known, for example, from U.S. Pat. No. 5,024,998, and references incorporated by reference therein. Other cyclodextrins suitable for use in the present invention include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. No. 6,610,671, 6,479,467, 6,660,804, or 6,509,323, each of which is specifically incorporated by reference herein.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e.g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, UK, 1999).

Suitable surface modifiers for use in the present invention are described in the art, for example, U.S. Pat. Nos. 5,145,684, 5,510,118, 5,565,188, and 6,264,922, each of which is specifically incorporated by reference herein. Examples of surface modifiers and/or surface stabilizers suitable for use in the present invention include, but are not limited to, hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, e.g., Tween 20™ and Tween 80™ (ICI Specialty Chemicals)), polyethylene glycols (e.g., Carbowax 3550™ and 934™ (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68™ and F108™, which are block copolymers of ethylene oxide and propylene oxide), poloxamines (e.g., Tetronic 908™, also known as Poloxamine 908, which is a tetrafunctional block copolymer derived from sequential addition of propylene oxide and ethylene oxide to ethylenediamine (BASF Wyandotte Corporation, Parsippany, N.J.)), Tetronic 1508™ (T-1508) (BASF Wyandotte Corporation), Tritons X-200, which is an alkyl aryl polyether sulfonate (Rohm and Haas), Crodestas F-100™, which is a mixture of sucrose stearate and sucrose distearate (Croda Inc.), p-isononylphenoxypoly-(glycidol), also known as Olin-10G™ or Surfactant 10™ (Olin Chemicals, Stamford, Conn.), Crodestas SL-40® (Croda, Inc.), and SA9OHCO, which is C₁₈H₃₇CH₂(—CON(CH₃)—CH₂(CHOH)₄(CH₂OH)₂ (Eastman Kodak Co.), decanoyl-N-methylglucamide, n-decyl-β-D-glucopyranoside, n-decyl-β-D-maltopyranoside, n-dodecyl β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-noyl-β-D-glucopyranoside, octanoyl-N-methylglucamide, n-octyl-β-D-glucopyranoside, octyl β-D-thioglucopyranoside, PEG-phospholipid, PEG-cholesterol, PEG-cholesterol derivative, PEG-vitamin A, PEG-vitamin E, lysozyme, random copolymers of vinyl pyrrolidone and vinyl acetate, and the like. (e.g. hydroxypropyl methylcellulose, hydroxypropylcellulose, polyvinylpyrrolidone, copolymers of vinyl acetate, vinyl pyrrolidone, sodium lauryl sulfate and dioctyl sodium sulfosuccinate).

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅ dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl(ethenoxy)₄ ammonium chloride or bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, Mirapol™ and ALKAQUAT™ (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides, imide azolinium salts, protonated quaternary acrylamides, methylated quaternary polymers, such as poly[diallyl dimethylammonium chloride] and poly-[N-methyl vinyl pyridinium chloride], and cationic guar.

In the context of the present invention, solubility enhancers include aqueous solutions formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

Furthermore, the processes for producing nanometer sized particles, including SCF, can permit selection of a desired morphology (e.g., amorphous, crystalline, resolved racemic) by appropriate adjustment of the conditions for particle formation during precipitation or condensation. As a consequence of selection of the desired particle form, extended release of the selected medicament can be achieved. These particle fabrication processes are used to obtain nanoparticulates that have high purity, low surface imperfections, low surface charges and low sedimentation rates. Such particle features inhibit particle cohesion, agglomeration and also prevent settling in liquid dispersions. Additionally, because processes such as SCF can separate isomers of certain medicaments, such separation could contribute to the medicament's enhanced activity, effectiveness as well as extreme dose reduction. In some instances, isomer separation also contributes to reduced side effects.

A preferred class of solubility enhancers are the sulfoalkyl ether cyclodextrin derivatives (SAE-CD derivatives), as set forth in WO 2005/065649, WO 2005/065435 and WO 2005/065651. In particular, it is considered advantageous to use a molar excess of solubility enhancer with respect to the corticosteroid. A particularly preferred class of SAE-CD derivatives are the SBE-β-CD compounds, such as SBE7-β-CD (Captisol®), which is available from CyDex, Inc., Lenexa, Kans. Other solubility enhancers that may be included in the solution include Polysorbate 80. Preferred concentrations of Polysorbate 80, when present, include 0.01% and less, 0.005% and less and 0.001% and less. In particular, compositions comprising an SAE-CD, such as SBE7-β-CD, and excluding Polysorbate 80, are preferred. In preferred embodiments, the corticosteroid solution also comprises an additional active ingredient, especially a water soluble active ingredient. One class of compounds that is preferably included in the solution are the water soluble fast-acting β2-agonists, such as albuterol.

In various embodiments the solubility enhancer is micronized.

In some embodiments, the solubility enhancer is a combination of two or more components. For example, the solubility enhancer may be a combination of a cyclodextrin such as such as SBE7-β-CD and a polyoxyethylene sorbitan monooleate such as polysorbate 80.

In some embodiments of the systems and methods described herein, a corticosteroid-containing aqueous solution is employed which further comprises at least one solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In yet other embodiments, the solubility enhancer can have a concentration (w/v) ranging from about 5% to about 10%. In a preferred embodiment, the solubility enhancer can have a concentration (w/v) ranging from about 1% to about 8.0% when the solubility enhancer is a cyclodextrin or cyclodextrin derivative.

III. Corticosteroid Solution

Provided herein are methods of manufacturing corticosteroid solutions which comprise at least one corticosteroid, at least one solubility enhancer, water and other optional ingredients. In some embodiments, the corticosteroid solution is manufactured by a process comprising the steps of (a) combining ingredients of the corticosteroid solution comprising as starting materials a corticosteroid, at least one solubility enhancer and water in a high sheer mixer and (b) homogenizing the ingredients for a homogenizing period.

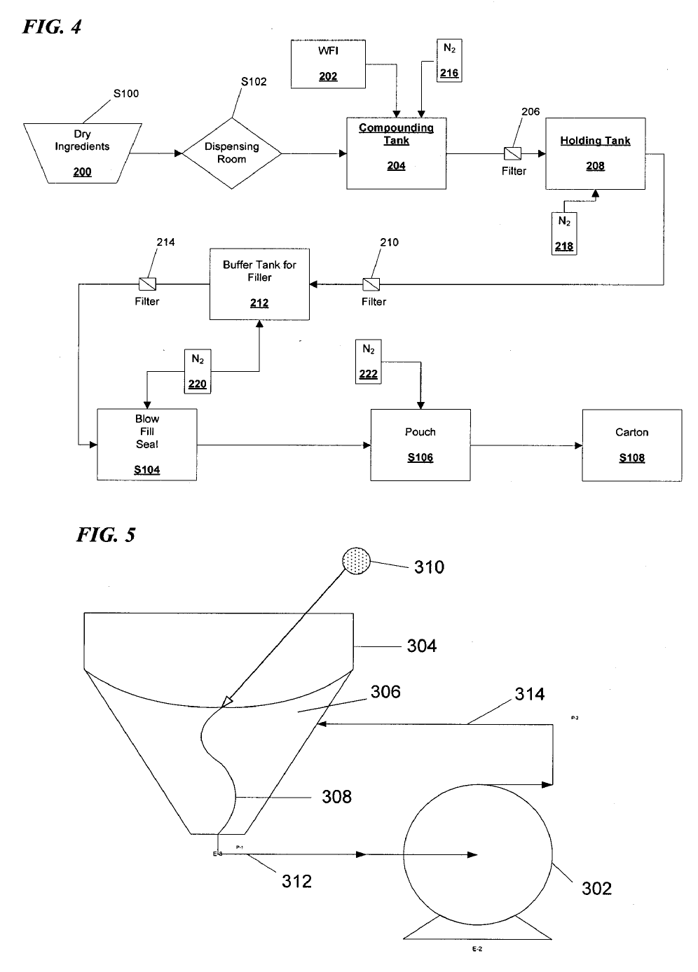

A process according to the present invention is illustrated in FIG. 4. (This is an illustrative, non-limiting embodiment; not all the illustrated steps are necessary in all embodiments of the invention.) In S100, dry ingredients 200 are identified and are assayed to determine their water content. Dry ingredients 200 include corticosteroid (e.g. budesonide, and particularly micronized budesonide) and cyclodextrin (e.g. Captisol® cyclodextrin), as well as additional ingredients, such as citric acid, sodium citrate, sodium chloride and sodium EDTA (sodium edetate). In S102, the ingredients 200 are moved to a dispensing room and are weighed and placed in containers suitable for dispensing the ingredients into the compounding tank 204. The cyclodextrin is advantageously divided into three aliquots; and the corticosteroid (e.g. budesonide) is placed in a suitable container. Water for injection (WFI) 202 is charged into the compounding tank 204. The dry ingredients 200 are then added to the compounding tank 204. At least a portion of the mixing in the compounding tank 204 is conducted under oxygen-depleted conditions. For example, the WFI 202 may have been sparged with nitrogen or argon to remove dissolved oxygen. Alternatively, the compounding tank 204 may be sealed and subjected to one or more (preferably two) cycles of vacuum/hold/overpressure with inert gas 216 (such as nitrogen or argon) during the mixing process. The overpressure of inert gas 216 may be a value above atmospheric pressure (any positive gauge pressure), and may for example be in the range of from 100 mbar to about 3000 mbar. In currently preferred embodiments, the overpressure is about 1,200 mbar of nitrogen gas. In some embodiments, the compounding tank 204 is fitted with a homogenization apparatus that is designed to create high shear conditions. In some embodiments, the compounding tank 204 is a FrymaKoruma Dinex® compounding mixer, which comprises a holding tank with a water jacket, an inlet for introducing liquid ingredients (e.g. WFI), a homogenizer, a stirrer, a short loop, a long loop and a funnel for introducing dry ingredients. High shear conditions in the FrymaKoruma Dinex® compounding mixer are approximately 1000 rpm to 4000 rpm, preferably about 1500 rpm to about 3000 rpm. For the 500 L batch size in a compounding tank 204 designed to accommodate a maximum volume of 500 L, one preferred homogenizer speed is about 2,500 rpm, although other values may be selected by one having skill in the art. For a 50 L batch size in a compounding tank 204 designed to accommodate a maximum volume of 500 L, one preferred homogenizer speed is about 1,700 rpm, although other values may be selected by one having skill in the art. The compounding tank 204 may be sealed to exclude atmospheric gasses. The compounding tank 204 may be any suitable size, in particular about 50 L to 1000 L capacity. The 500 L model is currently preferred. At the end of mixing (e.g. 30 to 600 min, and preferably about 120 min.) the corticosteroid (e.g. budesonide) solution is discharged under pressure into a holding tank 208. In some embodiments, a filter 206 is located between the compounding tank 204 and the holding tank 208. The filter may be a 0.1 to 0.22 μm pore diameter filter (preferably a 0.22 μm pore diameter) of a suitable composition (e.g. PVDF), e.g. a Millipore® CVGL71TP3 0.22 μm filter.

The corticosteroid (e.g. budesonide) solution may be held in the holding tank 208 for a period of time, e.g. up to seven days. The holding tank 208 may be air-tight and may be charged with an overpressure of inert gas 218, such as nitrogen or argon. In general, the inert gas pressure should be held well above atmospheric pressure, e.g. about 2000 mbar. The corticosteroid (e.g. budesonide) solution is next discharged under pressure into a buffer tank 212. The buffer tank 212 provides a mechanical buffer between the holding tank 208 and the filler in the Blow Fill Seal step S104. The buffer tank may also have a inert gas 220 overlay. A filter 210 may be interposed between the holding tank 208 and the buffer tank 212. When present, the filter 210 may be a 0.1 to 0.22 μm pore diameter filter (preferably a 0.22 μm pore diameter) of a suitable composition (e.g. PVDF), e.g. a Millipore® CVGL71TP3 0.22 μm filter.

The budesonide solution is discharged from the buffer tank 212 to a Blow Fill Seal apparatus in step S104. A filter 214 may be interposed between the buffer tank 212 and the Blow Fill Seal apparatus in step S104. When present, the filter 214 may be a 0.1 to 0.22 μm filter (preferably a 0.22 cm PVDF filter), e.g. a Millipore® CVGL71TP3 0.22 μm filter. The Blow Fill Seal step S104 entails dispensing the liquid corticosteroid (e.g. budesonide) solution into individual pharmaceutically acceptable containers (referred to elsewhere herein as bottles, ampoules or vials) and sealing the individual containers. In some embodiments, the containers are LDPE ampoules having a nominal capacity of 0.5 ml, although other materials and sizes are within the skill in the art. In some embodiments, the Blow Fill Seal step S104 may be conducted under oxygen-depleted conditions, such as positive inert gas 220 (e.g. nitrogen) pressure. The individual containers are then packaged in pouches in the Pouch step S106. In some embodiments, the Pouch step S106 may be carried out under oxygen-depleted conditions, such as under positive inert gas 222 (e.g. nitrogen) pressure. Each pouch may contain one or more containers (e.g. ampoules or vials) of corticosteroid (e.g. budesonide). In some embodiments, each pouch contains 1, 2, 3, 4, 5, 6, 7, 8, 9, 10, 11, 12, 13, 14 or more containers. In some currently preferred embodiments, each pouch contains 5 ampoules. The pouches are packaged into cartons in the Carton step S108.

In some embodiments, the corticosteroid solution is manufactured by mixing a mass of corticosteroid, solubility enhancer and other ingredients in a high sheer mixer for about 3 days, about 2 days, about 1 day, about 16 hours, about 12 hours, or about 8 hours.

In various embodiments, the corticosteroid solution is manufactured by mixing a mass of corticosteroid, solubility enhancer and other ingredients in a high sheer mixer for less than about 5, less than about 4, less than about 3 and in particular about 2 hours or less. In some embodiments, the mixing is conducted under nitrogen.

In some embodiments, the corticosteroid solution is manufactured by mixing a mass of corticosteroid, solubility enhancer and other ingredients in a high sheer mixer for between about 15 minutes to about 5 hours, or from between about 15 minutes to about 4 hours, or from between about 30 minutes to about 3 hours, or from between about 30 minutes to about 2 hours.

In some embodiments, the corticosteroid solution has at least about 90% dissolution after 5 minutes, or after 10 minutes, or after 15 minutes, or after 20 minutes, or after 25 minutes, or after 30 minutes of mixing. In other embodiments, the corticosteroid solution has at least about 95% dissolution after 5 minutes, or after 10 minutes, or after 15 minutes, or after 20 minutes, or after 25 minutes, or after 30 minutes of mixing. In some embodiments, the corticosteroid solution has at least about 98% dissolution after 5 minutes, or after 10 minutes, or after 15 minutes, or after 20 minutes, or after 25 minutes, or after 30 minutes of mixing.

In some embodiments, once mixing begins, the corticosteroid solution has at least about 98% dissolution within about 5 hours, or within about 4 hours, or within about 3 hours, or within about 2 hours, or within about 1 hour, or within about 30 minutes, or within about 15 minutes. In other embodiments, once mixing begins, the corticosteroid solution has at least about 95% dissolution within about 5 hours, or within about 4 hours, or within about 3 hours, or within about 2 hours, or within about 1 hour, or within about 30 minutes, or within about 15 minutes.

In some embodiments, between 15 minutes and 5 hours of mixing the corticosteroid solution achieves at least about 98% dissolution. In another embodiment, between about 15 minutes and 4 hours of mixing the corticosteroid solution achieves at least about 98% dissolution. In still other embodiments, between about 15 minutes and about 3 hours of mixing the corticosteroid solution achieves at least about 98% dissolution. In yet other embodiments, between about 30 minutes and about 1 hour of mixing the corticosteroid solution achieves at least about 98% dissolution.

In particular embodiments, the mixing is carried out in a high sheer mixer having a capacity of at least about 5 L, at least about 10 L, at least about 20 L, at least about 40 L, at least about 50 L, at least about 100 L, at least about 250 L, at least about 500 L, or at least about 1000 L. In some such preferred embodiments, the mixing is carried out with alternating cycles of vacuum and overlay with positive inert gas (such as N2 or Ar) pressure. In some specific embodiments, after mixing the solution is stored under an inert gas overlay (N2 or Ar) of at least about 100 mbar, at least about 200 mbar, at least about 500 mbar, at least about 1000 mbar, or about 1200 mbar or more.

In other embodiments, the mixing is carried out in a high sheer mixer having a capacity of between about 5 to 1000 L, or between about 25 to 1000 L, or between about 50 to about 1000 L, or between about 50 to about 700 L, or between about 50 to about 500 L, or between about 100 to about 500 L. In some such preferred embodiments, the mixing is carried out with alternating cycles of vacuum and overlay with positive inert gas (such as N₂ or Ar) pressure. In some specific embodiments, after mixing the solution is stored under an inert gas overlay (N₂ or Ar) of at least about 100 mbar, at least about 200 mbar, at least about 500 mbar, at least about 1000 mbar or about 1200 mbar or more.

In some embodiments, the corticosteroid solution has a volume of at least about S L, at least about 10 L, at least about 20 L, at least about 40 L, at least about 50 L, at least about 100 L, at least about 250 L, at least about 500 L, or at least about 1000 L. In some such preferred embodiments, the mixing is carried out with alternating cycles of vacuum and overlay with positive inert gas (such as N2 or Ar) pressure. In some specific embodiments, after mixing the solution is stored under an inert gas overlay (N2 or Ar) of at least about 100 mbar, at least about 200 mbar, at least about 500 mbar, at least about 1000 mbar or about 1200 mbar or more.

In various embodiments, the volume of the corticosteroid solution is between about 5 to 1000 L, or between about 25 to 1000 L, or between about 50 to about 1000 L, or between about 50 to about 700 L, or between about 50 to about 500 L, or between about 100 to about 500 L. In some such preferred embodiments, the mixing is carried out with alternating cycles of vacuum and overlay with positive inert gas (such as N₂ or Ar) pressure. In some specific embodiments, after mixing the solution is stored under an inert gas overlay (N₂ or Ar) of at least about 100 mbar, at least about 200 mbar, at least about 500 mbar, at least about 1000 mbar, or about 1200 mbar or more.

In addition to corticosteroid and solubility enhancers described above, the corticosteroid solution may include other active ingredients, especially other water-soluble active ingredients. Particularly suitable active ingredients are those that act either in conjunction with, or synergistically with, the corticosteroid for the treatment of one or more symptoms of pulmonary disease, such as bronchial spasm, inflammation of bronchia, etc. The corticosteroid thus may be compounded with one or more other drugs, such as β₂ adrenoreceptor agonists (such as albuterol), dopamine D₂ receptor antagonists, anticholinergic agents or topical anesthetics. Specific active ingredients are known in the art, and preferred embodiments are set forth on pages 48-49 of WO 2005/065651, which pages are expressly incorporated herein by reference in their entirety.

In some embodiments, other active ingredients, especially water soluble active ingredients are included in the corticosteroid solution. In some preferred embodiments, the corticosteroid solution includes a water soluble short acting β2-agonist, such as albuterol. Thus, some preferred embodiments include budesonide, a molar excess (relative to budesonide) of a cyclodextrin solubility enhancer, such as SBE7-β-CD, and albuterol.

Turboemulsifier

In alternative embodiments, dissolution of the active ingredient is achieved with a vacuum turboemulsifier, constituted by a steel container and fitted with a high-power turbine, and optionally used with an agitation system. The “high-power turbine” means a turbine with a power of between 15 to 55 Kwatts.

The vacuum turboemulsifier is constituted by a steel container, a high-power turbine, a hopper fitted inside an isolator and connected to the turbine of the turboemulsifier via a rigid pipe or hose, and optionally an agitation system. An “isolator” is a transparent container fitted with one or more entrance doors for transfer of the powder using handling gloves. The entry of the powder into the hopper can be regulated by a butterfly valve to minimize the introduction of air into the turboemulsifier.

In one embodiment, the turboemulsifier is the FrymaKoruma Dinex® 700 (FrymaKoruma GmbH, Neuenburg, DE) vacuum processor. In another embodiment, the vacuum turboemulsifier is one made by any number of companies including Charles Ross and Son Company, Pope Scientific, Inc., or RPA Process Technologies.

In a first step, the aqueous solution constituting the vehicle is prepared in a suitable tank. The solution can be sterilized not at all, or by heat or filtration, may be subjected to clarifying filtration, and may contain suitable additives or excipients, stabilizing agents and/or buffers. The solution thus obtained is transferred to a turboemulsifier with a vacuum pump. Alternatively, the aqueous solution can be prepared or sterilized in the turboemulsifier via a jacket that may be fitted onto the turboemulsifier which can steam heat or water cool the turboemulsifier.

In the second step, the active ingredient in solid, e.g., powder or crystal form is then either added from the top directly into the turboemulsifier or otherwise transferred through the turbine after applying the vacuum in the turboemulsifier.

In the third step, the active ingredient is homogenized under vacuum using the turbine system and operating between 750 and 4000 rpm, preferably between 1000 and 3600 rpm, and even more preferably between 1600 and 3000 rpm, for 5-60 minutes, and preferably for 20-40 minutes. In the preferred conditions a turbine system operating at 2900 rpm for 30 minutes is used. In some embodiments, a 50 L batch is homogenized at approximately 1700 rpm for e.g. about 2 hr. In some embodiments, a 500 L batch is homogenized at approximately 2500 rpm for e.g. about 2 hr.

High Speed Mixer

Another embodiment of the dissolution step according to the invention is depicted in FIG. 5, which is a schematic process flow diagram. A mixing vessel 304 contains the solution 306, which includes a portion of the WFI to be included in the final solution. The solution 306 is subjected to a vortex 308, e.g. using a high speed mixing apparatus (not shown). Budesonide 310 is introduced directly into the vortex as indicated by the arrow leading from the budesonide 310 to the top of the vortex 308. The solution 306 is drawn through pipe 312, through homogenizing pump 302 and re-circulated back into the mixing vessel 304 via the pipe 314. This recirculation and high speed mixing is effective to dissolved the budesonide 310 to form the final budesonide solution. Using a high speed mixer as depicted in FIG. 5, essentially any sized mixing tank can be accommodated with a homogenizing pump of appropriate capacity. Thus, batch sizes of 50 L, 500 L, 1000 L, 4000 L and 10,000 L or more may be accommodated using an apparatus as depicted in FIG. 5. In some embodiments, the homogenizing pump is an in-line high shear rotor/stator homogenizer.

### EXAMPLES

The following ingredients, processes and procedures for practicing the systems and methods disclosed herein correspond to that described above. Methods, materials, or excipients which are not specifically described in the following examples are within the scope of the invention and will be apparent to those skilled in the art with reference to the disclosure herein. The following examples as for exemplary purposes only and do not constitute the full scope of the present invention.

### Example 1A

### Dissolution Study-1A

The ingredients listed in Table 1A were used in dissolution study 1A. The solution was made by first preparing a solution containing the Captisol (“SBE7-β-CD” or “CAP”) and water. The water soluble ingredients were then added and the pH was adjusted to 4.5±0.5. The budesonide was then added to the solution and the suspension was stirred at room temperature for 5 hours. The total volume of the budesonide solution was 100 ml. The formulation was then filtered using a 0.22 μm filter. The filtered composition, representing dissolved budesonide, was compared to unfiltered budesonide, representing the total budesonide in the mixture. The results of dissolution study 1A are given in Table 1A-1.

TABLE 1A
(5.0/2.5/1.25 w % CAP).

| Ingredient | HIGH [w %] | MED [w %] | LOW [w %] |
|---|---|---|---|
| Budesonide | 0.048 | 0.024 | 0.012 |
| Captisol | 5.0 | 2.5 | 1.25 |
| Citric acid | 0.03 | 0.03 | 0.03 |
| Sodium citrate 2H₂O USP | 0.05 | 0.05 | 0.05 |
| NaCl | 0.37 | 0.60 | 0.71 |
| Na-EDTA 2H₂O | 0.01 | 0.01 | 0.01 |
| Water | ad 100.0 | ad 100.0 | ad 100.0 |

The results from the study are shown in Table 1A-1 below.

TABLE 1A-1
Results Of The Dissolution Study 1A

| | HIGH | | | MED | | | LOW | | |
|---|---|---|---|---|---|---|---|---|---|
| Time | BUD | BUD | | BUD | BUD | | BUD | BUD | |
| [h] | [μg/ml] | [%] | sd | [μg/ml] | [%] | sd | [μg/ml] | [%] | sd |
| 5 | 435.31 | 91.97 | 0.305 | 214.98 | 92.06 | 3.160 | 109.87 | 93.59 | 0.226 |
| unfiltrated | 473.30 | 100.00 | 1.917 | 233.51 | 100.00 | 0.149 | 117.40 | 100.00 | 0.396 |

### Example 1B

### Dissolution Study-1B

A solution containing the materials listed in Table 1B was made according to the procedure outlined in Example 1A. The filtered composition, representing dissolved budesonide, was compared to unfiltered budesonide, representing the total budesonide in the mixture. The results of dissolution study 1B are given in Table 1B-1.

TABLE 1B
(6.0/3.0/1.5 w % CAP).

| Ingredient | HIGH [w %] | MED [w %] | LOW [w %] |
|---|---|---|---|
| Budesonide | 0.048 | 0.024 | 0.012 |
| Captisol | 6.0 | 3.0 | 1.5 |
| Citric acid | 0.03 | 0.03 | 0.03 |
| Sodium citrate 2H₂O USP | 0.05 | 0.05 | 0.05 |
| NaCl | 0.28 | 0.55 | 0.685 |
| Na-EDTA 2H₂O | 0.01 | 0.01 | 0.01 |
| Water | ad 100.0 | ad 100.0 | ad 100.0 |

The results from the study are shown in Table 1B-1 below.

TABLE 1B-1
Results of Dissolution Study-1B

| | HIGH | | | MED | | | LOW | | |
|---|---|---|---|---|---|---|---|---|---|
| Time | BUD | BUD | | BUD | BUD | | BUD | BUD | |
| [h] | [μg/ml] | [%] | sd | [μg/ml] | [%] | sd | [μg/ml] | [%] | sd |
| 0 | 0.00 | 0.00 | 0.000 | 0.00 | 0.00 | 0.000 | 0.00 | 0.00 | 0.000 |
| 1 | 423.98 | 91.13 | 0.276 | 217.97 | 93.87 | 0.120 | 104.44 | 89.63 | 0.169 |
| 2 | 438.10 | 94.17 | 0.022 | 222.65 | 95.89 | 0.318 | 110.18 | 94.56 | 0.091 |
| 3 | 443.78 | 95.39 | 1.713 | 224.43 | 96.65 | 0.283 | 111.44 | 95.64 | 0.014 |
| 4 | 445.33 | 95.72 | 0.218 | 225.17 | 96.97 | 0.360 | 111.91 | 96.04 | 0.304 |
| 5 | 448.47 | 96.40 | 1.081 | 225.92 | 97.30 | 0.183 | 112.48 | 96.53 | 0.297 |
| 6 | 449.24 | 96.56 | 0.139 | 226.05 | 97.35 | 0.086 | 112.66 | 96.69 | 0.071 |
| 24 | 456.60 | 98.14 | 0.735 | 226.93 | 97.73 | 0.211 | 113.82 | 97.68 | 0.552 |
| unfiltrated | 465.24 | 100.00 | 0.219 | 232.20 | 100.00 | 0.276 | 116.52 | 100.00 | 0.184 |
| weighed | 482.6 | | | 244.9 | | | 121 | | |
| [μg/g] | | | | | | | | | |
| Density | 1.0243 | | | 1.0137 | | | 1.0085 | | |
| [g/ml] | | | | | | | | | |
| weighed | 494.3 | 106.25 | | 248.3 | 106.91 | | 122.0 | 104.73 | |
| [μg/ml] | | | | | | | | | |

### Example 1C

### Dissolution Study-1C

A solution containing the materials listed in Table 1C was made according to the procedure outlined in Example 1A. The filtered composition, representing dissolved budesonide, was compared to unfiltered budesonide, representing the total budesonide in the mixture.

TABLE 1C
(7.5/3.75/1.875 w % CAP).

| Ingredient | HIGH [w %] | MED [w %] | LOW [w %] |
|---|---|---|---|
| Budesonide | 0.048 | 0.024 | 0.012 |
| Captisol | 7.5 | 3.75 | 1.875 |
| Citric acid | 0.03 | 0.03 | 0.03 |
| Sodium citrate 2H₂O USP | 0.05 | 0.05 | 0.05 |
| NaCl | 0.145 | 0.483 | 0.651 |
| Na-EDTA 2H₂O | 0.01 | 0.01 | 0.01 |
| Water | ad 100.0 | ad 100.0 | ad 100.0 |

The results from study 1C are shown in Table 1C-1 below.

TABLE 1C-1
Results of Dissolution Study 1-C

| | HIGH | | | MED | | | LOW | | |
|---|---|---|---|---|---|---|---|---|---|
| Time | BUD | BUD | | BUD | BUD | | BUD | BUD | |
| [h] | [μg/ml] | [%] | sd | [μg/ml] | [%] | sd | [μg/ml] | [%] | sd |
| 0 | 0.00 | 0.00 | 0.313 | 0.00 | 0.00 | 0.000 | 0.00 | 0.00 | 0.000 |
| 1 | 325.70 | 65.40 | 0.313 | 170.20 | 70.58 | 0.071 | 73.11 | 61.81 | 0.014 |
| 2 | 461.17 | 92.60 | 1.204 | 226.42 | 93.89 | 0.163 | 106.82 | 90.31 | 0.191 |
| 3 | 464.21 | 93.21 | 0.409 | 228.62 | 94.80 | 0.014 | 110.80 | 93.68 | 0.092 |
| 4 | 468.24 | 94.02 | 0.084 | 230.98 | 95.78 | 0.199 | 112.12 | 94.79 | 0.346 |
| 5 | 472.70 | 94.92 | 0.567 | 232.64 | 96.47 | 0.093 | 113.32 | 95.81 | 0.403 |
| 6 | 476.39 | 95.66 | 0.043 | 234.45 | 97.22 | 0.155 | 114.30 | 96.64 | 0.085 |
| 24 | 493.57 | 99.11 | 0.296 | 238.10 | 98.74 | 0.360 | 116.05 | 98.11 | 0.057 |
| unfiltrated | 498.02 | 100.00 | 0.762 | 241.15 | 100.00 | 0.289 | 118.28 | 100.00 | 0.361 |
| weighed | 488.8 | N.D. | N.D. | 238.6 | N.D. | N.D. | 120.6 | N.D. | N.D. |
| [μg/g] | | | | | | | | | |
| Density | 1.0297 | N.D. | N.D. | 1.0164 | N.D. | N.D. | 1.0099 | N.D. | N.D. |
| [g/ml] | | | | | | | | | |
| weighed | 503.3 | 101.06 | N.D. | 242.5 | 100.57 | N.D. | 121.8 | 102.97 | N.D. |
| [μg/ml] | | | | | | | | | |

### Example 1D

### Dissolution Study-1D

A solution containing the materials listed in Table 1D was made according to the procedure outlined in Example 1A. The filtered composition, representing dissolved budesonide, was compared to unfiltered budesonide, representing the total budesonide in the mixture. The results of dissolution study 1D are given in Table 1D-1.

TABLE 1D
HIGH/LOW formulations with PS80.

| | HIGH | HIGH | LOW | LOW |
|---|---|---|---|---|
| | 6.0/0.01 | 7.5/0.01 | 1.5/0.02 | 1.875/0.01 |
| Ingredient | [w %] | [w %] | [w %] | [w %] |
| Budesonide | 0.048 | 0.048 | 0.012 | 0.012 |
| Captisol | 6.0 | 7.5 | 1.5 | 1.875 |
| Polysorbate 80 | 0.01 | 0.01 | 0.02 | 0.01 |
| Citric acid | 0.03 | 0.03 | 0.03 | 0.03 |
| Sodium citrate | 0.05 | 0.05 | 0.05 | 0.05 |
| 2H₂O USP | | | | |
| NaCl | 0.28 | 0.145 | 0.685 | 0.651 |
| Na-EDTA 2H₂O | 0.01 | 0.01 | 0.01 | 0.01 |
| Water | ad 100.0 | ad 100.0 | ad 100.0 | ad 100.0 |

The results from the study are shown in Table 1D-1, below.

TABLE 1D-1
Results of Dissolution Study 1D

| | HIGH 6.0% CAP | | | HIGH 7.5% CAP | | | LOW 1.5% CAP | | | LOW 1.875% CAP | | |
|---|---|---|---|---|---|---|---|---|---|---|---|---|
| | 0.01% PS80 | | | 0.01% PS80 | | | 0.02% PS80 | | | 0.01% PS80 | | |
| Time | BUD | BUD | | BUD | BUD | | BUD | BUD | | BUD | BUD | |
| [h] | [μg/ml] | [%] | sd | [μg/ml] | [%] | sd | [μg/ml] | [%] | sd | [μg/ml] | [%] | sd |
| 0 | 0.00 | 0.00 | 0.000 | 0.00 | 0.00 | 0.000 | 0.00 | 0.00 | 0.000 | 0.00 | 0.00 | 0.000 |
| 1 | 460.69 | 92.60 | 0.447 | 480.10 | 97.56 | 0.302 | 120.04 | 96.98 | 0.120 | 122.65 | 99.02 | 0.056 |
| 2 | 466.01 | 93.67 | 0.149 | 486.60 | 98.88 | 0.141 | 122.57 | 99.02 | 0.148 | 123.89 | 100.02 | 0.134 |
| 3 | 468.66 | 94.20 | 0.197 | 486.19 | 98.80 | 0.214 | 122.85 | 99.25 | 0.071 | 124.07 | 100.16 | 0.035 |
| 4 | 468.88 | 94.24 | 0.628 | 484.95 | 98.55 | 0.567 | 122.51 | 98.97 | 0.198 | 123.90 | 100.02 | 0.021 |
| 5 | 469.61 | 94.39 | 0.615 | 486.09 | 98.78 | 0.870 | 122.80 | 99.21 | 0.254 | 124.19 | 100.26 | 0.176 |
| 24 | 476.92 | 95.86 | 0.291 | 495.59 | 100.71 | 0.129 | 124.27 | 100.40 | 0.078 | 125.01 | 100.92 | 0.106 |
| unfiltrated | 497.52 | 100.00 | 0.214 | 492.09 | 100.00 | 0.044 | 123.78 | 100.00 | 0.219 | 123.87 | 100.00 | 0.042 |
| weighed | 487.1 | N.D. | — | 480.2 | N.D. | — | 125.6 | N.D. | — | 126.2 | N.D. | — |
| [μg/g] | | | | | | | | | | | | |
| Density | 1.0243 | N.D. | — | 1.0297 | N.D. | — | 1.0085 | N.D. | — | 1.0099 | N.D. | — |
| [g/ml] | | | | | | | | | | | | |
| weighed | 498.9 | 100.28 | — | 494.5 | 100.48 | — | 126.7 | 102.33 | — | 127.4 | 102.96 | — |
| [μg/ml] | | | | | | | | | | | | |

### Example 1E

### Dissolution Study-1E

A solution containing the materials listed in Table 1E was made according to the following procedure. A solution of water and captisol was made and stirred using a magnetic stirrer. The water-soluble ingredients were then added and the pH was adjusted to 4.5±0.5. Budesonide was added to the solution and the suspension was stirred until the budesonide was dispersed. Further dispersion of budesonide was accomplished by using an Ultra-Turrax (20 min stirring/20 min cooling in refrigerator). The solution was then filtered using a 0.2 μm filter. The filtered composition, representing dissolved budesonide, was compared to unfiltered budesonide, representing the total budesonide in the mixture. The results of dissolution study 1E are given in Table 1E-1.

TABLE 1E
formulations with PS80.

| Ingredient | MED [w %] |
|---|---|
| Budesonide | 0.024 |
| Captisol | 3.0 |
| Citric acid | 0.03 |
| Sodium citrate 2H₂O USP | 0.05 |
| NaCl | 0.55 |
| Na-EDTA 2H₂O | 0.01 |
| Water | ad 100.0 |

The results from the study are shown in Table 1E-1 below.

TABLE 1E-1
Results of Dissolution Study 1E

| | MED | | |
|---|---|---|---|
| Time | BUD | BUD | |
| [min] | [μg/ml] | [%] | sd |
| 60 | 224.74 | 96.83 | 0.135 |
| 100 | 228.46 | 98.44 | 0.354 |
| unfiltrated | 232.09 | 100.00 | 0.346 |

### Example 2

### Preparation of 120 Microgram/Milliliter Budesonide Solution

A 50 L batch of budesonide solution (nominally 120 μg/ml) was prepared according to the following procedure:

Prior to weighing the Captisol® cyclodextrin (Cyclodextrin) and budesonide, the starting materials were assayed. The assay values were used to calculate the actual amount of Cyclodextrin and budesonide starting materials to be used in the formulation. The Cyclodextrin was found to be 4.9% water (95.1% Cyclodextrin). Thus, the total amount of Cyclodextrin starting material was increased by a proportional amount. It was calculated that the amount of Cyclodextrin starting material needed was 935.8569 g (representing 890.0 g Cyclodextrin). This Cyclodextrin starting material was weighed out in three measure: 735.86 g, 100.0 g and 100.0 g. In the same way, the budesonide starting material was assayed and found to contain 98.2% budesonide base. The amount of budesonide starting material was then calculated to be 5.95 g/0.982=6.06 g. Thus, 6.06 g of budesonide starting material was weighed out.

The following additional ingredients were weighed out: 15.0 g citric acid anhydrous; 25.0 g sodium citrate dihydrate USP. Sufficient water for injection to make up 50 kg of solution was also provided.

The mixing apparatus comprised a high sheer mixer a feed funnel in an isolator, as well as a vacuum apparatus and a source of nitrogen gas. The high sheer mixer was enclosed, thereby making it possible to apply a vacuum to the contents of the mixer during mixing.

40 kg of water were introduced into to a mixing apparatus (FrymaKouma Dinex® vacuum processor, 500 L max volume). A 224 mbar vacuum was taken on the mixing apparatus and held for 5 minutes. Then 1278 mbar (gauge pressure) of nitrogen gas was introduced into the mixing vessel, which remained isolated from atmosphere outside the mixer during the duration of the mixing procedure. About one third of the Captisol® cyclodextrin was added to the funnel in the isolator. Then about 100.0 g of Cyclodextrin was added to the budesonide starting material in an Erlenmeyer flask and shaken until a homogeneous mixture was formed. This mixture was then added to the feed funnel. Then 100.0 g of Cyclodextrin was added to the Erlenmeyer flask and shaken until homogeneous. The contents of the Erlenmeyer flask were then added to the funnel. Finally 15.0 g citric acid anhydrous, 25.0 sodium citrate dihydrate USP, 5.0 g sodium EDTA dihydrate and 325.0 g sodium chloride were each sequentially added to the funnel. When all the ingredients had been combined in the funnel, all were introduced to the mixer by vacuum suction.

The contents of the mixer were then homogenized at 1500 rpm for about 5 minutes at about 17° C. The Erlenmeyer flask that formerly contained the budesonide starting material was then rinsed twice with about 150 ml water; and the rinse water was added to the funnel. Abut half of the remaining water was added to the funnel and the contents of the funnel were introduced into the mixer by vacuum suction. Then the final quantity of water was added to the funnel and introduced into the mixer by vacuum suction. Finally, the homogenizer speed was increased to 1700 rpm for 120 minutes.

During the 120 minute homogenization, the mixing tank was purged of oxygen as follows: (1) A first vacuum of about 200 mbar was applied and held for about 5 minutes; (2) a nitrogen pressure of 1200 mbar was applied; (3) a second vacuum of about 200 mbar was applied and held for about 5 minutes; and (4) a second nitrogen overlay of about 1215 mbar was applied to the mixer. At the end of homogenization, samples of the homogenized budesonide solution were taken and sent to Q.C.

### Example 3

### Dissolution Study-3

TABLE 2
Budesonide Solution From Dissolution Study-3

| | Nominal | Nominal | Actual |
|---|---|---|---|
| Ingredient | [w %] | [g]¹ | [g]¹ |
| Micronized Budesonide | 0.0236 | 11.8 | 11.8 |
| Captisol (contains 4.9 wt-% water) | 3.75 | 1875.0 | 1875.0 |
| Citric acid, anhydrous | 0.03 | 15.0 | 15.0 |
| Sodium citrate, 2H₂O USP | 0.05 | 25.0 | 25.0 |
| Sodium chloride | 0.49 | 245.0 | 245.0 |
| Disodium edetate, 2H₂O USP | 0.01 | 5.0 | 5.0 |
| Water | 95.6464 | 47823.2 | 48300.0 |

| | |
|---|---|
| BUD (Actual) [μg/g] | 233.8 |
| Density [g/ml] | 1.017 |
| BUD (Actual) [μg/ml] = 100% | 237.7 |

¹50 kg batch

The following procedure was used in this dissolution study-3: (1) Water for injection (41.0 kg) was added to the Dinex 700 processing unit. (2) Vacuum (200 mbar) was applied. Nitrogen was applied at 1200 mbar. The processes were repeated. (3) The weighed dry ingredients were placed in the eccentric addition funnel which was placed under a glove box. The addition occurs in a sequence of increasing weights whereby the emptied plastic vessel of budesonide was filled twice with a portion of Captisol, closed, shaken and emptied into the funnel to remove rests of budesonide. The funnel was closed with a lid. (4) A portion of 6.8 kg water for injection was added to funnel and used to flush powder from the funnel's surface to the bottom. (5) The velocity of the homogenizer was adjusted to 1700 min⁻¹. (5) The powder is scraped to the bottom of the funnel by use of a rubber spatula. A portion of 500 ml water for injection was used to flush powder to the bottom. The funnel was closed with a lid. (6) Sterile air was let in the Dinex 700 processing unit to adjust atmospheric pressure. (a) Stirring process/sampling. (i) T=50 min (T₀=1^{st} suction process) (7) A sample (budesonide assay) was taken from the loop, filtered through a 0.45 μm filter. (8) Vacuum (200 mbar) was applied. Nitrogen was applied at 1200 mbar. The processes were repeated. (i) T=60 min (T₀=1^{st} suction process) (9) Samples were taken from the loop (filtered 0.45 μm) and from the top of the vessel (filtered 0.45 μm, unfiltered). The stirring process was thereby stopped for 10-15 min. (i) T=120-360 min (T₀=1^{st} suction process) (10) Samples were taken from the loop (filtered 0.45 μm, unfiltered) every hour. (11) After 360 min additional unfiltered samples were taken from the loop and the top of the vessel. (13) The product was transferred into a non-sterile holding tank and discarded afterwards.

TABLE 3
Assay results (BUD = budesonide)

| | filtered (loop)³ | | | filtered (top)³ | | | unfiltered⁴ | | |
|---|---|---|---|---|---|---|---|---|---|
| Time | BUD | BUD | | BUD | BUD | | BUD | BUD | |
| [min]² | [μg/ml] | [%]⁵ | sd | [μg/ml] | [%]⁵ | sd | [μg/ml] | [%]⁵ | sd |
| 50 | 238.6 | 100.4 | 0.8 | N.D. | N.D. | — | N.D. | N.D. | — |
| 60 | 229.1 | 96.4 | 0.3 | 228.4 | 96.1 | 0.2 | 228.2 | 96.0 | 0.1 |
| 120 | 229.1 | 96.4 | 0.4 | N.D. | N.D. | — | 231.6 | 97.4 | 0.2 |
| 180 | 229.2 | 96.4 | 0.3 | N.D. | N.D. | — | 231.0 | 97.2 | 0.1 |
| 240 | 229.9 | 96.7 | 0.4 | N.D. | N.D. | — | 231.2 | 97.2 | 0.2 |
| 300 | 230.2 | 96.8 | 0.5 | N.D. | N.D. | — | 231.9 | 97.5 | 0.4 |
| 360 | 229.0 | 96.3 | 0.1 | N.D. | N.D. | — | 230.1 | 96.8 | 0.2 |
| end loop | N.D. | N.D. | — | N.D. | N.D. | — | 231.1 | 97.2 | 1.0 |
| end top | N.D. | N.D. | — | N.D. | N.D. | — | 228.4 | 96.1 | 0.1 |

²T₀ = 1^{st} suction process
³0.45 μm Methylcellulose filter
⁴60 min: top, others: loop
⁵100% = 237.7 μg/ml BUD = theoretical BUD concentration
N.D. = Not Determined

### Example 4

### Dissolution Study-4

A similar process as that described in Example 3 was used except with the following materials.

TABLE 4
Budesonide Solution for Dissolution Study-4

| | Nominal | Nominal | Actual |
|---|---|---|---|
| Ingredient | [w %] | [g]¹ | [g]¹ |
| Micronized Budesonide | 0.0236 | 11.8 | 11.8 |
| Captisol (contains 4.9 wt-% water) | 3.75 | 1875.0 | 1875.0 |
| Citric acid, anhydrous | 0.03 | 15.0 | 15.0 |
| Sodium citrate, 2H₂O USP | 0.05 | 25.0 | 25.0 |
| Sodium chloride | 0.49 | 245.0 | 245.0 |
| Disodium edetate, 2H₂O | 0.01 | 5.0 | 5.0 |
| Water | 95.6464 | 47823.2 | 47800.0 |

| | |
|---|---|
| BUD (Actual) [μg/g] | 236.1 |
| Density [g/ml] | 1.0175 |
| BUD (Actual) [μg/ml] = 100% | 240.2 |

The results are shown in Table 4-1 below.

TABLE 4-1
Results of Dissolution Study 4

| | filtered³ | | | unfiltered | | |
|---|---|---|---|---|---|---|
| Time | BUD | | | BUD | BUD | |
| [min]² | [μg/ml] | BUD [%]⁵ | sd | [μg/ml] | [%]⁵ | sd |
| 30 | 234.7 | 97.7 | 0.6 | N.D. | N.D. | — |
| 60 | 235.9 | 98.2 | 0.7 | N.D. | N.D. | — |
| 90 | 237.4 | 98.8 | 0.1 | N.D. | N.D. | — |
| 120 | 236.9 | 98.6 | 1.5 | N.D. | N.D. | — |
| 150 | 237.8 | 99.0 | 0.1 | N.D. | N.D. | — |
| 180 | 237.7 | 98.9 | 0.1 | N.D. | N.D. | — |
| 210 | 237.2 | 98.7 | 0.8 | N.D. | N.D. | — |
| 240 | 237.2 | 98.7 | 1.1 | 239.2 | 99.6 | 1.0 |
| holding tank⁴ | 237.8 | 99.0 | 0.2 | N.D. | N.D. | — |

²T₀ = 1^{st} suction process
³0.45 μm Methylcellulose filter
⁴0.2 μm PVDF filter
⁵100% = 240.2 μg/ml BUD = theoretical BUD concentration

### Example 5

### Dissolution Study-5 (50 kg Batch)

TABLE 5
A similar process as that described in Example 3 was used
except with the following materials.

| | Nominal | Nominal | Actual |
|---|---|---|---|
| Ingredient | [w %] | [g]¹ | [g]¹ |
| Micronized Budesonide | 0.0466 | 23.3 | 23.3 |
| Captisol (4.9 wt-% water) | 7.5 | 3750.0 | 3750.0 |
| Citric acid, anhydrous | 0.03 | 15.0 | 15.0 |
| Sodium citrate, 2 H₂O USP | 0.05 | 25.0 | 25.0 |
| Sodium chloride | 0.15 | 75.0 | 75.0 |
| Disodium edetate, 2 H₂O | 0.01 | 5.0 | 5.0 |
| Water | 92.2134 | 46106.7 | 46100.0 |

| | |
|---|---|
| BUD (Actual) [μg/g] | 466.1 |
| Density [g/ml] | 1.0307 |
| BUD (Actual) [μg/ml] = 100% | 480.4 |

¹50 kg batch

The results are shown in Table 5-1, below.

TABLE 5-1
Results of Dissolution Study 5

| | filtered³ | | | unfiltered | | |
|---|---|---|---|---|---|---|
| | BUD | | | BUD | | |
| Time | [μg/ml] | BUD [%]⁴ | sd | [μg/ml] | BUD [%]⁴ | sd |
| 30 | 471.6 | 98.2 | 1.0 | N.D. | N.D. | — |
| 60 | 475.7 | 99.0 | 2.6 | N.D. | N.D. | — |
| 90 | 472.9 | 98.5 | 0.2 | N.D. | N.D. | — |
| 120 | 475.4 | 99.0 | 0.6 | N.D. | N.D. | — |
| 150 | 472.5 | 98.4 | 1.2 | N.D. | N.D. | — |
| 180 | 473.5 | 98.6 | 0.6 | 478.8 | 99.7 | 0.3 |

²T₀ = 1^{st} suction process
³0.45 μm Methylcellulose filter
⁴100% = 480.4 μg/ml BUD = theoretical BUD concentration

### Example 6

### Dissolution Study-6

TABLE 6
A similar process as that described in Example 3 was used
except with the following materials.

| Ingredient | Nominal [w %] | Nominal [g]¹ | Actual [g]¹ |
|---|---|---|---|
| Micronized Budesonide | 0.0236 | 11.8 | 11.8 |
| Captisol (4.9 wt-% water) | 3.75 | 1875.0 | 1875.0 |
| Citric acid, anhydrous | 0.03 | 15.0 | 15.0 |
| Sodium citrate, 2H₂O USP | 0.05 | 25.0 | 25.0 |
| Sodium chloride | 0.49 | 245.0 | 245.0 |
| Disodium edetate, 2H₂O | 0.01 | 5.0 | 5.0 |
| Water | 95.6464 | 47823.2 | 47800.0 |

| | |
|---|---|
| BUD (Actual) [μg/g] | 236.1 |
| Density [g/ml] | 1.0176 |
| BUD (Actual) [μg/ml] = 100% | 240.3 |

¹50 kg batch

The results are shown in Table 6-1 below.

TABLE 6-1
Results of Dissolution Study 6

| | filtered³ | | | unfiltered | | |
|---|---|---|---|---|---|---|
| Time | BUD | | | BUD | | |
| [min]² | [μg/ml] | BUD [%]⁴ | sd | [μg/ml] | BUD [%]⁴ | sd |
| 30 | 232.6 | 96.8 | 0.7 | N.D. | N.D. | — |
| 60 | 232.9 | 96.9 | 0.8 | N.D. | N.D. | — |
| 90 | 234.5 | 97.6 | 0.1 | N.D. | N.D. | — |
| 120 | 235.1 | 97.8 | 0.3 | 237.0 | 98.6 | 0.9 |

²T₀ = last suction process
³0.45 μm Methylcellulose filter
⁴100% = 240.3 μg/ml BUD = theoretical BUD concentration

### Example 7

### 80 Microgram/Milliliter Budesonide Solution (Batch G1059)

A 50 L batch of budesonide solution having a final concentration of approximately 80 μg/ml was prepared according to the following procedure.

First, budesonide and Captisol® cyclodextrin (Cyclodextrin) were assayed to determine the percent water in each sample. The target mass of cyclodextrin in the 50 L batch was 595 g; and the target mass of budesonide was 4.1 g. The assay for Cyclodextrin gave a value of 4.8% water or 95.2% Cyclodextrin; the budesonide assay gave a percent budesonide value of 99.2%. Thus, the amount of Cyclodextrin was calculated to be 595 g/0.952=625 g Cyclodextrin; the budesonide mass was calculated to be 4.1 g/0.992=4.133 g budesonide.

The cyclodextrin was weighed out in three aliquots of 100 g, 100 g and 425 g of cyclodextrin, respectively. 4.133 g of budesonide were weighed out in a container (budesonide container).

A cleaned holding tank was steam sterilized and 40 kg of water for injection (WFI) were charged into the holding tank. A clean stainless steel 500 L (max capacity) FrymaKoruma Dinex® (FrymaKoruma GmbH, Neuenburg, Germany) mixing vessel (mixing tank) with a stirrer and homogenizer was steam sterilized for 10 minutes and dried. The mixing tank is equipped with a short homogenization loop (short loop) and a funnel for introduction of dry ingredients (dry-addition funnel; funnel). The 40 kg of water were then transferred to the mixing tank from the holding tank under pressure. Approximately half of the pre-weighed 425 g aliquot of Cyclodextrin were then added to the dry-addition funnel. The entire contents of the budesonide container were then added to the funnel, taking care not to allow any of the budesonide to contact the walls of the funnel. The first 100 g aliquot of Cyclodextrin was then added to the budesonide container and shaken to scavenge any residual budesonide. The contents of the budesonide container were then added to the funnel. This procedure was repeated with the second 100 g aliquot of Cyclodextrin.

The following quantities of ingredients were then added to the funnel: 15.0 of anhydrous citric acid, 25.0 g of sodium citrate dihydrate, 5.0 g sodium edetate dihydrate, 395.0 g of sodium chloride and the second half of Cyclodextrin from the 425 g aliquot. With the stirrer set to 25 rpm and the homogenizer set to 1500 rpm, the entire contents of the dry funnel were added to the mixing tank under suction. The contents of the mixing tank were then homogenized through the short loop for approximately 10 minutes.

The budesonide container was then washed with two 150 g aliquots of WFI: A first 150 g aliquot of WFI was added to the budesonide container and shaken. The contents of the budesonide container were then added to the funnel. This procedure was repeated with a second 150 g aliquot of WFI and then the entire contents (˜300 ml) of the funnel were added to the mixing tank by suction. Approximately half of 8.631 kg of WFI was added to the funnel. The WFI in the funnel was then added to the mixing tank by suction. This procedure was repeated with the remaining approximately half of the 8.631 kg of WFI.

The homogenizer speed was increased to 1700 rpm. The mixing tank was then purged with nitrogen (N₂): A vacuum of −200 mbar was applied to the mixing tank and held for five minutes; then the mixing tank was pressurized with 1,200 mbar of nitrogen. This procedure was repeated once. Samples of budesonide solution were drawn from the mixing tank through a 0.22 μm PVDF filter at 60, 90 and 120 minutes. At the end of 124 minutes, the entire contents of the mixing tank were discharged through Teflon® PTFE hose and a 0.22 μm Durapore® PVDF cartridge filter and into a holding tank. The procedure netted 46.6 kg of 80.2 μg/ml (assay value) budesonide solution. The budesonide solution was blow filled into LDPE vials under nitrogen to produce filled vials containing 0.53 ml/vial (42.1 μg/vial of budesonide). The sealed LDPE vials were pouched—five vials per pouch—under nitrogen. Each solution passed sterility according to USP <71> and PhEur 2.6.1.

### Example 8

### 40, 60, 120 and 240 μg/0.5 mL Dose Budesonide Solutions

Following the general procedures outlined in Examples 1 and 7, above, budesonide solutions having concentrations of 80, 120, 240 and 480 μg/mL were prepared, dispensed into LDPE vials (ampoules) in 0.5 mL doses and pouched as described above. The resulting 0.5 mL doses contained 40, 60, 120 and 240 μg budesonide per 0.5 mL dose. The amounts of each ingredient contained in each ampoule are set forth in the Table, below. Each solution passed sterility according to USP <71> and PhEur 2.6.1.

TABLE 7
40, 60, 120 and 240 μg/0.5 mL Dose Budesonide

| | 240 mcg/ | 120 mcg/ | 60 mcg/ | 40 mcg/ |
|---|---|---|---|---|
| Ingredient | 0.5 mL | 0.5 mL | 0.5 mL | 0.5 mL |
| Budesomde | 0.048 | 0.024 | 0.012 | 0.008 |
| Captisol | 7.5 | 3.57 | 1.78 | 1.19 |
| Citric acid | 0.03 | 0.03 | 0.03 | 0.03 |
| Sodium citrate | 0.05 | 0.05 | 0.05 | 0.05 |
| dihydrate USP | | | | |
| NaCl | 0.45 | 0.57 | 0.73 | 0.79 |
| Na-EDTA | 0.01 | 0.01 | 0.01 | 0.01 |
| Water | ad 100.0 | ad 100.0 | ad 100.0 | ad 100.0 |

### Values shown are [w %]; Osmolality adjusted to 290 mOsm/kg; pH 4.5

### Example 9

### Budesonide Dissolution—Comparison of Factors

In order to determine the effect of various parameters on the manufacturing efficiency of budesonide solutions according to the invention, several batches of budesonide were prepared essentially as described in Example 7, above, with the modifications (dissolution factors) listed in following Table 8 below. In short, four factors—2 levels each—were analyzed: Scale (50 L or 500 L); Homogenizer Speed (50 kg-1700 rpm±200 rpm; 500 kg-2500 rpm±200 rpm); Temperature: 15-20° C. or 30-35° C.; and budesonide concentration: 120 μg/mL or 240 μg/mL. Each solution passed sterility according to USP <71> and PhEur 2.6.1.

TABLE 8
Dissolution Factors

| | Scale | Homogenizer Speed, | Temperature | Concentration |
|---|---|---|---|---|
| Run | kg | rpm | ° C. | μg/mL |
| 1 | 50 | 1500 | 30–35 | 240 |
| 2 | 50 | 1500 | 15–20 | 120 |
| 3 | 500 | 2700 | 15–20 | 120 |
| 4 | 50 | 1900 | 30–35 | 120 |
| 5 | 500 | 2700 | 30–35 | 240 |
| 6 | 500 | 2300 | 15–20 | 240 |
| 7 | 500 | 2300 | 30–35 | 120 |
| 8 | 50 | 1900 | 15–20 | 240 |

The conditions for each of the dissolution trials is listed below in Table 9.

TABLE 9
Manufacturing Details and Compounding Data.

| | | Batch # | | | | | | | | | | |
|---|---|---|---|---|---|---|---|---|---|---|---|---|
| | | GE086 | GE088 | GE089 | GE090 | GE099 | GE109 | GE119 | GE123 | GE129 | GE150 | GE166 |
| Batch | Target conc, | 240 | 120 | 120 | 120 | 240 | 240 | 120 | 240 | 120 | 120 | 120 |
| Descr | [ug/mL] | | | | | | | | | | | |
| | Scale, [kg] | 50 | 50 | 500 | 50 | 500 | 500 | 500 | 50 | 50 | 50 | 50 |
| | Homogenizer | 1500 | 1500 | 2700 | 1900 | 2700 | 2300 | 2300 | 1900 | 1700 | 1700 | 1700 |
| | speed, [rpm] | | | | | | | | | | | |
| | Temperature, [C.] | 30–35 | 15–20 | 15–20 | 30–35 | 30–35 | 15–20 | 30–35 | 15–20 | 15–25 | 15–25 | 15–25 |
| Comp | Captisol, incl. | 1859 | 927 | 9271 | 927 | 18594 | 18594 | 9271 | 1859 | 927 | 935 | 756 |
| | water [g] | | | | | | | | | | | |
| | Budesonide [g] | 12.4 | 6.3 | 62.7 | 6.3 | 124.4 | 124.4 | 62.7 | 12.4 | 6.3 | 6.2 | 6.3 |
| | Citric Acid, | 15 | 15 | 150 | 15 | 150 | 150 | 150 | 15 | 15 | 15 | 15 |
| | anhydrous [g] | | | | | | | | | | | |
| | Sodium citrate | 25 | 25 | 250 | 25 | 250 | 250 | 250 | 25 | 25 | 25 | 25 |
| | 2H₂O, USP [g] | | | | | | | | | | | |
| | Sodium chloride | 265 | 345 | 3450 | 345 | 2650 | 2650 | 3450 | 265 | 345 | 345 | 345 |
| | [g] | | | | | | | | | | | |
| | Disodium | 5 | 5 | 50 | 5 | 50 | 50 | 50 | 5 | 5 | 5 | 5 |
| | edetate 2H₂O [g] | | | | | | | | | | | |
| | Water for | 47.8 | 48.7 | 486.8 | 48.7 | 478.2 | 478.2 | 486.8 | 47.8 | 48.7 | 48.7 | 48.7 |
| | injection [kg] | | | | | | | | | | | |
| Yield | Yield [kg] | 46.2 | 46.8 | 491.5 | 47.0 | 493.0 | 492.5 | 492.0 | 46.6 | 47.2 | 46.2 | 48.7 |
| | Yield [%] | 92% | 94% | 98% | 94% | 99% | 98% | 98% | 93% | 94% | 92% | 98% |

Comp = Composition

The dissolution profiles for the above lots is set forth in Table 10 below. Budesonide solution was extracted from each batch through a 0.22 μm PVDF filter at time points of 60, 90, 120, 150 minutes of mixing and the concentration of dissolved budesonide was determined by HPLC.

TABLE 10
Dissolution Profiles

| | Batch # | | | | | | | | | | |
|---|---|---|---|---|---|---|---|---|---|---|---|
| | GE086 | GE088 | GE089 | GE090 | GE099 | GE109 | GE119 | GE123 | GE129 | GE150 | GE166 |
| BUD assay | 224.7 | 112.6 | 116.0 | 113.0 | 233.3 | 234.9 | 116.8 | 223.1 | 111.7 | 112.3 | 113.2 |
| 60 min | | | | | | | | | | | |
| [μg/mL] | | | | | | | | | | | |
| BUD assay | 229.8 | 1121 | 115.5 | 112.7 | 233.1 | 234.9 | 117.9 | 226.7 | 111.3 | 112.3 | 112.7 |
| 90 min | | | | | | | | | | | |
| [μg/mL] | | | | | | | | | | | |
| BUD assay | 228.9 | 111.6 | 115.7 | 114.2 | 236.5 | 235.2 | 117.7 | 224.1 | 112.2 | 112.2 | 112.4 |
| 120 min | | | | | | | | | | | |
| [μg/mL] | | | | | | | | | | | |
| BUD assay | 95.4 | 93.0 | 96.4 | 95.2 | 98.5 | 98.0 | 98.1 | 93.4 | 93.5 | 93.5 | 93.7 |
| 120 min | | | | | | | | | | | |
| [% nominal] | | | | | | | | | | | |
| BUD assay | 228.3 | 110.3 | 116.0 | 113.3 | 234.0 | 234.5 | 117.9 | 226.2 | na | na | na |
| 150 min | | | | | | | | | | | |
| [μg/mL] | | | | | | | | | | | |
| BUD assay | 223.3 | 112.3 | 114.2 | filled | filled | 232.8 | 117.8 | 220.6 | filled | filled | filled |
| hold tank | | | | | | | | | | | |
| [μg/mL] | | | | | | | | | | | |
| pH, bulk | 4.46 | 4.47 | 4.45 | 4.44 | 4.44 | 4.43 | 4.46 | 4.43 | 4.47 | 4.42 | 4.46 |
| Osmolality | 279 | 276 | 282 | 276 | 281 | 273 | 287 | 279 | 277 | 278 | 277 |
| [mOsmol/kg], | | | | | | | | | | | |
| bulk | | | | | | | | | | | |
| Density | 1.018 | 1.011 | 1.011 | 1.011 | 1.019 | 1.018 | 1.012 | 1.018 | 1.011 | 1.011 | 1.011 |
| [g/cm³], bulk | | | | | | | | | | | |

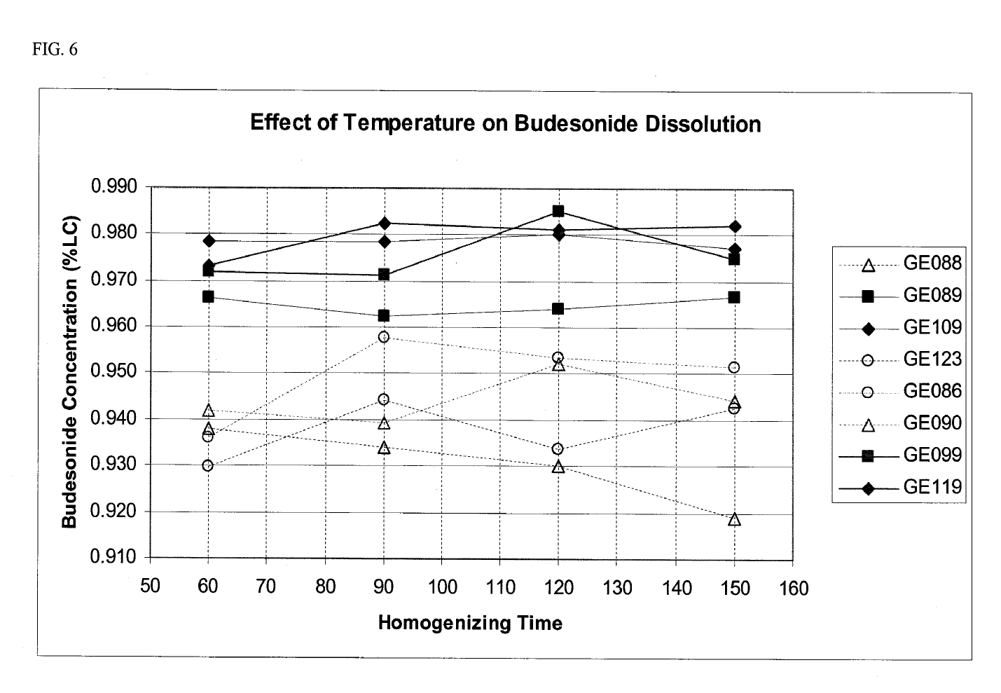

Tukey-Kramer analysis of the foregoing data (generated with JMP 5.1.2, SAS Institute, Cary, N.C., USA) is summarized in Table 11 below. As can be seen, the one factor having the greatest impact on the outcome of the dissolution studies was “Scale.” This effect was statistically significant: P<0.05 for all time points. 50 kg batches were 3-4% lower than 500 kg batches. Temperature, on the other hand, showed statistically significant effect at 120 minutes (p=0.03). There was 2% increased budesonide dissolution at 30-35° C. at 120 minutes. The temperature results are shown in FIG. 6. This trend was less pronounced at 90 and 15 minutes. However, there was no significant trend seen for homogenizer speed under these conditions.

TABLE 11

| | P Value | P Value | P Value | P Value | | | |
|---|---|---|---|---|---|---|---|
| | BUD | BUD | BUD | BUD | P Value | P Value | P Value |
| Factor | 60 min | 90 min | 120 min | 150 min | pH | Osmol | Density |
| Scale | 0.004 | 0.007 | 0.003 | 0.01 | 0.64 | 0.43 | 0.18 |
| Homogenizer Speed | 0.43 | 0.14 | 0.58 | 1.0 | 0.22 | 0.85 | 1.0 |
| Temperature | 0.61 | 0.19 | 0.03 | 0.20 | 0.64 | 0.43 | 0.18 |
| Concentration | 0.92 | 0.15 | 0.21 | 0.31 | 0.22 | 0.58 | 0.0002 |

Budesonide dissolution data for two batch scales (50 and 500 kg, respectively) and two temperature ranges (15-20° C. and 30-35° C., respectively) are set forth in Table 12 below. Two separate temperature scales were chosen: 15-20° C. (ambient) and 30-35° C. (elevated). As can be seen in FIG. 9, there was a pronounced influence on dissolution rate at elevated temperatures at the 120 minute mark.

TABLE 12
Budesonide Dissolution Data for Two Temperatures and Two Batch Sizes

| | GE088 | GE089 | GE109 | GE123 | GE086 | GE090 | GE099 | GE119 |
|---|---|---|---|---|---|---|---|---|
| Target Bud Conc | 120 | 120 | 240 | 240 | 240 | 120 | 240 | 120 |
| [μg/ml] | | | | | | | | |
| Scale [kg] | 50 | 500 | 500 | 50 | 50 | 50 | 500 | 500 |
| Temperature [° C.] | 15–20 | 15–20 | 15–20 | 15–20 | 30–35 | 30–35 | 30–35 | 30–35 |
| BUD assay 60 min | 0.938 | 0.966 | 0.979 | 0.930 | 0.936 | 0.942 | 0.972 | 0.973 |
| [% LC] | | | | | | | | |
| BUD assay 90 min | 0.934 | 0.963 | 0.979 | 0.944 | 0.958 | 0.939 | 0.971 | 0.982 |
| [% LC] | | | | | | | | |
| BUD assay 120 min | 0.930 | 0.964 | 0.980 | 0.934 | 0.954 | 0.952 | 0.985 | 0.981 |
| [% LC] | | | | | | | | |
| BUD assay 150 min | 0.919 | 0.967 | 0.977 | 0.943 | 0.951 | 0.944 | 0.975 | 0.982 |
| [% LC] | | | | | | | | |

Although preferred embodiments of the present invention have been shown and described herein, it will be apparent to those skilled in the art that such embodiments are provided by way of example only. Numerous variations, changes, and substitutions will be apparent to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered herein.

What is claimed is:

1. A process of making a corticosteroid solution, comprising the steps of:
(a) combining ingredients of the corticosteroid solution comprising as starting materials a corticosteroid, at least one solubility enhancer and water in a high sheer mixer having a capacity greater than or equal to about 50 L; and
(b) homogenizing the ingredients for a homogenizing period of about 5 hours or less;
whereby at least about 98% of the corticosteroid starting material is dissolved within the homogenizing period.

2. The process of claim 1, wherein the corticosteroid is budesonide.

3. The process of claim 1, wherein the solubility enhancer comprises a sulfoalkyl ether cyclodextrin (SAE-CD).

4. The process of claim 3, wherein the SAE-CD is SBE7-β-CD.

5. The process of claim 1, wherein the corticosteroid solution further comprises albuterol.

6. The process of claim 1, wherein at least about 98.5% of the corticosteroid is dissolved within the homogenizing period.

7. The process of claim 1, wherein the homogenizing period is about 2 hours or less.

8. The process of claim 1, wherein at least about 99% of the corticosteroid is dissolved within the homogenizing period.

9. The process of claim 8, wherein the homogenizing period is about 2 hours or less.

10. The process of claim 1, wherein at least about 99.5% of the corticosteroid is dissolved within the homogenizing period.

11. The process of claim 10, wherein the homogenizing period is about 2 hours or less.

12. The process of claim 1, wherein at least about 95% of the corticosteroid is dissolved within the first hour of the homogenizing period.

13. The process of claim 12, wherein at least about 97% of the corticosteroid is dissolved within the first hour of the homogenizing period.

14. The process of claim 1, wherein the high sheer mixer has a capacity of about 100 L to about 10000 L, about 250 L to about 4000 L or about 500 L.

15. The process of claim 1, wherein the budesonide solution substantially excludes polysorbate 80.

16. The process of claim 1, wherein the budesonide solution contains less than about 0.01 wt-% polysorbate 80 or less than about 0.005 wt-% polysorbate 80.

17. The process of claim 1, wherein the budesonide solution comprises two or more solubility enhancers.

18. The process of claim 17, wherein the solubility enhancer is a combination of polyoxyethylene sorbitan monooleate and a cyclodextrin.

19. The process of claim 18, wherein the polyoxyethylene sorbitan monooleate is polysorbate 80.

20. The process of claim 19, wherein the polysorbate is present in an amount of between about 0.005 wt-% to about 0.1 wt-%.

21. The process of claim 1, wherein the high sheer mixer is a FrymaKoruma Dinex model 700, 1300, 2400, 3500, 4200 or 5200.

22. The process of claim 21, wherein the high sheer mixer is a FrymaKoruma Dinex model 700.

23. The process of claim 1, wherein the homogenization speed is between about 1000 to about 3000 rpm.

24. The process of claim 23, comprising homogenizing the mixture at a homogenization speed of about 1500 to about 3000 rpm.

25. The process of claim 24, wherein the homogenization speed is about 1700 rpm to about 2500 rpm.
